(19) United States
(12) Patent Application Publication
(10) Pub. No.: US 2010/0104610 A1
(43) Pub. Date: Apr. 29, 2010

(54) Color cosmetics comprising a random terpolymer

(76) Inventors: Olga V. Dueva-Koganov, White Plains, NY (US); Bingham S. Jaynes, New City, NY (US); Joseph A. Lupia, Monroe, NY (US); Arjan T. Termaten, Assen (NL); Marcelles Van der Sluis, Groningen (NL); Marleen Suurmeijer, Joure (NL); David Normington, West Yorkshire (GB); Marcel Schnyder, Birsfelden (CH)

Correspondence Address: Ciba Corporation;Patent Department, 540 White Plains Road, P.O. Box 2005, Tarrytown, NY, 10591, US

(21) Appl. No.: 12/566,273

(22) Filed: Sep. 24, 2009

Related U.S. Application Data
(60) Provisional application No. 61/194,877, filed on Oct. 1, 2008.

(51) Int. Cl. A61K 8/18; A61Q 1/02; A61Q 3/00; A61Q 1/04; A61Q 1/00; A61Q 1/10

(52) U.S. Cl. 424/401; 424/63; 424/61; 424/64; 424/70.6; 424/70.7

(57) ABSTRACT

Topically applicable, water-resistant cosmetic or dermatological compositions well suited for the cosmetic color compositions for human skin and/or hair comprising at least one
(a) coloring agent;
(b) at least one random terpolymer; and, optionally
(c) other cosmetically acceptable ingredients.

This application claims the benefit of U.S. provisional application No. 61/194,877, filed on Oct. 1, 2008.

### FIELD OF THE INVENTION

Pigmented cosmetic compositions containing a random terpolymer impart the pigmented cosmetic with long lasting adherence to the skin, high resistance to water permeability and resistance to smudging, smearing and transfer of said compositions. Furthermore, the pigment cosmetic compositions containing said terpolymer provide highly dispersive properties to the pigments which in turn gives smoother more even application of colored films on skin and hair. The pigmented compositions include such cosmetic products as foundation makeup, lipstick, lip gloss, mascara, eyeliner, eye shadow and the like.

### BACKGROUND OF THE INVENTION

Pigmented cosmetic compositions such as foundation makeup, blush, lipstick, mascara, eyeliner and eyeshadow, are used to color the skin and lips. Since color is one of the most important reasons for wearing cosmetics, color containing cosmetics must be very carefully formulated to provide maximum wear and effect.

One of the long standing problems with color cosmetics such as face makeup, lipstick, mascara, and the like, is the tendency of the cosmetic to blot or transfer from the skin or lashes onto other surfaces such as glassware, silverware, or clothing. This not only creates soiling, but forces the cosmetic user to reapply cosmetic at fairly short intervals.

Traditional makeup compositions are either water and oil emulsions containing pigments, or they can be anhydrous systems containing waxes, oils and pigments. These formulations are applied and blended into the skin or hair such as lashes and eyebrows to provide color and correct skin topography to provide an even, smooth appearance. The films are deposited on the surface of the skin or hair and if touched with fingers the product may transfer or become blotchy and uneven. Perspiration or sebum will break through the film and cause running or smearing. If skin comes into contact with clothing, the clothing may become soiled.

Various cosmetic products, such as loose or compact powders, make-up foundations, blushes, eye shadows, lipsticks and lip glosses are colored using organic and inorganic pigments dispersed in a carrier.

The formulated cosmetic products may be oil-in-water emulsions, water-in-oil emulsions or anhydrous compositions. Further, many cosmetic or dermatological products comprise a structured, i.e., gelled and/or rigidified, liquid fatty phase, such as, for example, in mascaras, lipsticks, concealer products, eyeshadows, and foundations. This structuring may be obtained with the aid of traditional waxes and/or fillers. Unfortunately, these waxes and fillers may have a tendency to make the composition matte and to dull the intensity and color of any pigments in the composition, which may not always be desirable, in particular for a mascara. Specifically, consumers are always on the lookout for a mascara, for example which can deposit a film with intense color and which is also increasingly glossy.

While the formulated color cosmetic may be oil-in-water or water-in-oil, oil-in-water is generally preferred because of the better sensory characteristics of oil-in-water formulations. Water-in-oil formulations have a tendency to feel somewhat greasy while oil-in-water have a lighter less occlusive feel on skin.

Despite the desirable properties of oil-in-water emulsions, it is difficult to prepare these emulsions while maintaining even dispersion of organic and/or inorganic pigments within the emulsions. This is because finely divided pigments especially metal oxide pigments, tend to agglomerate. Specifically, the pigment particles attract to one another and form a colloid or enlarged clump of pigment when added directly to oil-in-water dispersions. If the pigments are not evenly dispersed, aesthetically unpleasing dark spots or swirls may appear in the final cosmetic. Further, the uneven dispersions or agglomeration of pigment particles creates an abrasive feel on the skin. Further they are subject to smudging once they come into contact with water or perspiration.

Both the intensity of the color and the gloss of a cosmetic composition are generally associated with the nature of the liquid fatty phase. The liquid fatty phase of mascaras commonly comprise a traditional wax. Traditional waxes do not develop pigments, and adding pigments to such traditional waxes generally results in a composition having a grey, dull color and a matte look.

Various attempts have been made to overcome the above problems in formulating color cosmetics.

For example, water resistant polymers are known in the cosmetic arts and their use lessens the loss of active ingredients in cosmetic formulations containing sunscreens. For example, there are numerous patents and applications which discuss water insoluble film forming materials for use in sunscreens. Co-pending U.S. Ser. Nos. 12/079,607 and 12/079,606 herein incorporated entirely by reference disclose waterproof sunscreens compositions which incorporate a terpolymer. These compositions significantly protect the sunscreen on skin from water erosion.

Additionally, U.S. Pat. Nos. 4,663,157, 6,409,998, 6,312,672 and 7,153,494 teach the use of water resistant or water insoluble polymers in combination with sunscreens.

U.S. Application Publication No. 2003/0021847 teaches water resistant polymer compositions having functional network structures which aid in retention of active ingredients on skin.

Furthermore there are many patents and publications which discuss dispersants for pigments in industrial coatings and printing inks. For example, U.S. Pat. No. 5,688,858, incorporated entirely by reference, discloses a multifunctional polymer suitable as a dispersant for industrial coatings and printing inks.

The inventors have discovered a novel solution for achieving two important effects in color cosmetics. By incorporation of a random terpolymer of formula (I) described herein, even dispersion of coloring agents onto skin and hair and water resistance of the applied color films on skin and hair are achieved.

### SUMMARY OF THE INVENTION

Therefore, a first aspect of the present invention is directed to a color cosmetic composition comprising at least one coloring agent, preferably a pigment,

at least one random terpolymer of formula (I), and other cosmetically acceptable ingredients.

A second aspect of the present invention is directed to a method of preparing a color cosmetic composition comprising mixing together at least one coloring agent, preferably a pigment,

at least one random terpolymer of formula (I) and, optionally, other cosmetically acceptable ingredients.

A third aspect of the present invention is directed to a method of increasing the smudge resistance of a cosmetic color composition on skin or hair wherein said method comprises incorporating into said compositions an effective amount of at least one random terpolymer according to formula (I) in combination with a coloring agent, preferably a pigment.

The term “effective amount” means for example the amount necessary to achieve the desired effect. The desired effect may be water, smudge and transfer resistance or adequate dispersion of coloring agent.

A fourth aspect of the present invention is directed to a method of improving the water resistance of a color cosmetic on skin or hair which method comprises applying to said skin and/or said hair the color cosmetic composition comprising at least one coloring agent, an effective amount of at least one random terpolymer of formula (I), and, optionally, other cosmetically acceptable ingredients.

Another object of the invention is to formulate a color cosmetic which yields a film which does not readily transfer to clothing or utensils.

Another object of the invention is directed to a topically applicable dermatological, eyebrow or eyelash color cosmetic composition comprising at least one coloring agent and a terpolymer of formula (I).

And finally, an object of the invention is to provide cosmetic color formulations of well dispersed organic and inorganic pigments which maintain an even coloring effect on skin and hair.

### DETAILED DESCRIPTION OF THE INVENTION

The present invention is directed to a cosmetic color composition comprising:

(a) at least one coloring agent, preferably a pigment,
(b) at least one random terpolymer of formula (I)

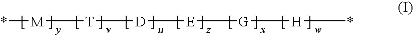

wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (II)

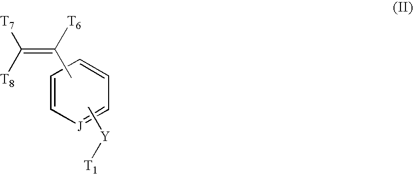

wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III)

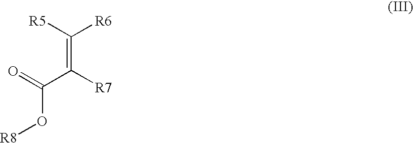

wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more —OH and/or NH2 groups; or said alkyl or said cycloalkyl may be interrupted by one or more —O— groups and/or N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-dilsocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile;
and
(c) other cosmetically acceptable ingredients,
with the proviso that T, D, and E are different from each other.

### Color Cosmetics

A color cosmetic for purposes of the invention means any color cosmetic formulation which color formulation may be applied as a film onto skin or hair.

The color cosmetic may be a solid, liquid, gelled, foamed product or stick.

The present color cosmetics are particularly suitable for coloration of skin or hair products, in particular:
cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, and nail-care preparations, such as nail varnish or colorants;
light-protective preparations, such as sun milks, lotions, creams or oils, sunblocks or tropicals, pre-tanning preparations or after-sun preparations;
skin-tanning preparations, e.g. self-tanning creams;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, hair foams, hairsprays, temporary, semi-permanent or permanent hair colourants, or natural hair colourants, such as henna or camomile;
decorative preparations, in particular lipsticks, nail varnishes, eye shadows, mascaras, dry and moist make-up, rouge and powders.

### Presentation Forms

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a stick,
in the form of a spray (spray with propellent gas or pump-action spray) or an aerosol,
in the form of a foam, or
in the form of a paste.

Examples of color cosmetic products of the present invention are listed in the Table below:

| | |
|---|---|
| Lipstick | Vegetable oils, waxes, stabilizers, dyes/pigments |
| Eye shadow | Talc, Zinc Stearate, oils, stabilizers, pigments |
| Makeup | Water, thickener, oils, emulsifier, perfume, preservatives, |
| | stabilizers, pigments |

The color cosmetics may be provided, in particular, in the form of a simple or complex (O/W, W/O, O/W/O or W/O/W) emulsion such as a cream, a milk, a gel or a gel cream, of a powder, a lotion, an ointment, a solid stick and may optionally be packaged as an aerosol and provided in the form of a foam, mousse or spray.

When an emulsion is provided, the aqueous phase thereof may comprise a nonionic vesicular dispersion prepared according to known techniques (Bangham, Standish and Watkins, J. Mol. Biol., 13, 238 (1965), FR-2,315,991 and FR-2,416,008).

The color cosmetic formulation will preferably contain water. The water content is for example at least 1, 2, 3, 4 or 5 wt % based on the total formulation. More typically the water content will be at least 10, 20 or 30 wt. %. Water ranges may vary for examples from about 10 to about 90 wt. % water, from about 10 to about 80 wt. % water, and more typically about 15 to about 75 wt. % water.

If the composition is an oil-in-water phase for example, the water makes up the continuous phase and the oil makes up the discontinous phase (oil droplets dispersed in the water).

### Coloring Agent

The coloring agent of the color cosmetic product, will for example contain one or more pigments, which may be organic, inorganic, or a combination thereof. Examples of useful pigments include, but are not limited, inorganic pigments such as iron oxides (yellow, red, brown or black), ferric ammonium ferrocyanide (blue), manganese violet, ultramarine blue, chrome oxide (green), talc, lecithin modified talc, zeolite, kaolin, lecithin modified kaolin, titanium dioxide (white) and mixtures thereof. Other useful pigments are pearlescents such as mica, bismuth oxychloride and treated micas, such as titanated micas and lecithin modified micas.

For example, specific inorganic pigments include:

Alumina, chromium-cobalt-aluminum oxide, ferric ammonium citrate, calcium carbonate, Iron oxides (synthetic and natural), ferric ammonium ferrocyanide, chromium hydroxide green, bismuth oxychloride, chromium oxide green, mica, titanium dioxide, aluminum powder, bronze powder, copper powder, ultramarines, manganese violet and zinc oxide.

Pearlescent pigments are well known inorganic pigments and are commonly known as effect or interference pigments and are also well known for use in cosmetics.

To achieve a pearlescent effect, interference pigments are used for cosmetic applications. Mica, coated with varying thickness of titanium dioxide has been used to yield a pigment with a silvery, pearl-like effect. See, e.g. U.S. Pat. No. 3,087,829 and U.S. Pat. No. 3,123,490. Later teachings disclosed the use of thin film optics that resulted in pigments with brilliant luster and a broad range of interference colors and multicolour effect. See, e.g. U.S. Pat. No. 6,132,873 and U.S. Pat. No. 4,323,544.

Interference pigments have been developed for color cosmetics and skin care to provide luster and color effect. See, e.g. JP11193215, WO9924001, and WO200174979.

Interference pigment comprise a multilayer structure. The core of the interference pigment is a flat substrate with a refractive index (R1) normally below 1.8. A wide variety of substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof.

A layer of thin film or a multiple layer of thin films are coated onto the surface of the substrate described above. The thin films are made of highly refractive materials. The refractive index of these materials is normally above 1.8.

A wide variety of thin films are useful. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures. Multiple layered are common which comprises high refractive index materials or alternation of thin films with high and low RI materials.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine.

Nonlimiting examples of the interference pigments available commercially and useful herein include those supplied by Persperse, Inc. under the trade name PRESTIGE, FLONAC; supplied by EMD Chemicals, Inc. under the trade name TIMIRON, COLORONA, DICHRONA, and XIRONA; supplied by Engelhard Co. under the trade name FLAMENCO, TIMICA, DUOCHROME and supplied by Ciba Corp. under the tradename CALISHA.

Useful organic pigments include natural colorants and synthetic monomeric and polymeric colorants. Exemplary organic pigments are phthalocyanine blue and green pigments, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C lakes and blends thereof.

Specific examples of organic FD&C approved pigments are:

### FD&C Blue No. 1 (CI No. 42090),

### FD&C Green No, 3 (CI No. 42053),

### FD&C Red No. 3 (CI No. 45430),

### FD&C Red No. 40 (CI No. 16035),

### FD&C Yellow No. 5 (CI No. 19140),

### FD&C Yellow No. 6 (CI No. 15985),

### D&C Blue No. 4 (CI No. 42090),

### D&C Green No. 5 (CI No. 61570),

### D&C Green No. 6 (CI No. 61565),

### D&C Yellow No. 10 (CI No. 47005),

### Ext D&C Yellow No. 7 (CI No, 10315),

### D&C Brown No. 1 (CI No. 20170),

### D&C Orange No. 4 (CI No, 15510),

### D&C Orange No. 5 (CI No. 45425),

### D&C Orange 17 (CI No. 12075),

### D&C Red No. 6 (CI No. 15850),

### D&C Red No. 7 (CI No. 15850),

### D&C Red No, 8 (CI No. 15585),

### D&C Red No. 9 (CI No. 15585),

### D&C Red No. 17 (CI No. 26100),

### D&C Red No. 19 (CI No. 45170),

### D&C Red No. 21 (CI No. 45380A),

### D&C Red No. 27 (CI No. 45410A),

### D&C Red No. 28 (CI No. 45410),

### D&C Red No. 30 (CI No. 73360),

### D&C Red No. 31 (CI No. 15800),

### D&C red No. 33 (CI No. 17200),

### D&C Red No. 34 (CI No. 15880),

### D&C Red No, 36 (CI No. 12085),

### D&C red No. 37 (CI No. 45170B),

### D&C Violet No. 2 (CI No. 60725),

### Ext D&C Violet No. 2 (CI No. 60730),

D&C Lakes-stratight colorant extended on substratum of alumina, blanc fixe, gloss, white, clay, titanium dioxide, zinc oxide, talc, rosin, aluminum benzoate, calcium carbonate, or any combination of two or more of these,
Ext. D&C Lakes-stratight colorant extended on substratum of alumina, blanc fixe, gloss, white, clay, titanium dioxide, zinc oxide, talc, rosin, aluminum benzoate, calcium carbonate, or any combination of two or more of these and mixtures thereof.

Also envisioned as coloring agents with the random terpolymer of the invention are polymer pigments, e.g., nylon powder, polyethylene, and polyesters. The polyesters can include linear, thermoplastic, crystalline or amorphous materials produced using one or more dials and one or more dicarboxylic acids copolymerized with colorants. Other pigments to be used in the invention will be apparent to one of ordinary skill in the art.

Representative inorganic pigments may also include for example nanopigments (mean size of the primary particles: generally between 5 nm and 100 nm, preferably between 10 nm and 50 nm) formed from coated or uncoated metal oxides, such as, for example, titanium oxide (amorphous or crystalline in the rutile and/or anatase form), iron oxide, zinc oxide, zirconium oxide or cerium oxide nanopigments.

There is no particular size limitation to the coloring agent or pigment particle but may be in the nanometer range as suggested above or larger particles such as 0.0001 to 500 microns and preferably 0.01 to 200 micrometers. The size is based on the widest diameter of the pigment particle.

A number of difficulties may arise in uniformly dispersing pigment particles, especially finely divided pigments. Untreated, many pigments, for example metal oxides such as iron oxide, titanium dioxide and zinc oxide, have significant surface reactivity which may be attributable to chemical reactivity either covalent or ionic, or to more physical phenomena such as adsorbability or accumulation of surface charge. Such surface reactivity may interfere with the uniformity of an initial dispersion of the powder or may adversely impact the long-term stability of the end product. The pigment particles may tend to couple covalently or electrochemically with other ingredients in the formulation or to agglomerate, which is to say to stick to each other in agglomerations or clumps. The result may be a poor or unacceptable end product or a product which has limited shelf life owing to non-uniformity of color or other properties, agglomeration, a poor, gritty or sandy feel, settling and so on.

Additionally surface tension and the hydrophilic nature of untreated inorganic pigments complicate incorporation into lipophilic systems, while strong oil absorption results in a negative impact on viscosity.

Finally untreated pigment particles, especially light particles of low density, cause serious dusting problems during handling of bulk material.

To overcome these problems, it has long been customary to surface treat pigments to render them hydrophobic and to enhance the handling, processing and performance of the pigment particles in finished products. Typical coatings work by reducing the surface activity of the pigment particles, repelling water or other aqueous media, inhibiting agglomeration, reducing oil absorption and enhancing dispersibility of the powder particles in aqueous or oily media used in formulating finished products. A satisfactory coating should cover each particle completely and more or less uniformly.

To these ends, many hydrophobic coatings and treatments are commercially available and have been proposed in the literature, especially in the patent literature. For example U.S. Application Publication No. 2008/0213322, herein incorporated entirely by reference, teaches a hydrophobic coated pigment.

Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

It is for example, known to coat pigments such as titanium dioxide and iron oxides with methicone, alginic acid, polyperfluoromethylisopropyl ether, stearic acid, stearyl triethoxysilane, triethoxycaprylylsilane, PEG-8 dimethicone, galactoarabinan and sodium C14-16 olefin sulfonate. Thus such coated pigments are useful in cosmetic color formulations in combination with the random terpolymer of formula (I).

The coloring agent will for examples make up about 0.01 to about 80 wt. % or 90 wt. % of the color cosmetic formulation. Essentially anhydrous formulations such as pressed face powder may contain major amounts of pigment whereas emulsion may contain lesser wt. % of pigments. For example, about 0.01 to about 15, 20, 30, 40 or 50 wt. % coloring agent is envisioned in an o/w or w/o emulsion.

### Random Terpolymer

The random copolymers of component b) formula (I), u+v+w+x+y+z=100 weight percent relative to the total weight of the terpolymer.

The random terpolymers of component (b) formula (I) according to the instant invention are derived from at least three different monomers. Another aspect of the instant invention is the random terpolymers of component (b) formula (I) are derived from at least four different monomers.

The random terpolymers of component (b) formula (I) can be used in conjunction with other polymers or copolymer in a color cosmetic formulation; for example, the polymers listed in U.S. Pat. No. 6,409,998 and/or in US 2006/0104923.

Another embodiment of the instant invention for component b) formula (I) is that y is from about 0.1% to about 35% by weight based on the total weight of the terpolymer. Another embodiment of the instant invention for component b) formula (I) is that y is from about 1% to about 30% by weight based on the total weight of the terpolymer. Another embodiment of the instant invention for component b) formula (I) is that y is from about 5% to about 20% by weight based on the total weight of the terpolymer.

Another embodiment of the instant invention for component b) formula (I) is that v is from about 5% to about 70% by weight based on the total weight of the terpolymer. Another embodiment of the instant invention for component b) formula (I) is that v is from about 5% to about 60% by weight based on the total weight of the terpolymer. Another embodiment of the instant invention for component b) formula (I) is that v is from about 10% to about 60% by weight based on the total weight of the terpolymer.

Another embodiment of the instant invention for component b) formula (I) is that u is from about 5% to about 75% by weight based on the total weight of the terpolymer. Another embodiment of the instant invention for component b) formula (I) is that u is from about 5% to about 65% by weight based on the total weight of the terpolymer. Another embodiment of the instant invention for component b) formula (I) is that u is from about 5% to about 60% by weight based on the total weight of the terpolymer.

Another embodiment of the instant invention for component b) formula (I) is that z is from about 0.1% to about 50% by weight based on the total weight of the terpolymer. Another embodiment of the instant invention for component b) formula (I) is that z is from about 1% to about 50% by weight based on the total weight of the terpolymer. Another embodiment of the instant invention for component b) formula (I) is that z is from about 1% to about 40% by weight based on the total weight of the terpolymer.

Another embodiment of the instant invention for component b) formula (I) is that x is from about 1% to about 40% by weight based on the total weight of the terpolymer. Another embodiment of the instant invention for component b) formula (I) is that x is from about 1% to about 30% by weight based on the total weight of the terpolymer. Another embodiment of the instant invention for component b) formula (I) is that x is from about 5% to about 25% by weight based on the total weight of the terpolymer.

Another embodiment of the instant invention for component b) formula (I) is that w is from about 0.1% to about 45% by weight based on the total weight of the terpolymer. Another embodiment of the instant invention for component b) formula (I) is that w is from about 1% to about 40% by weight based on the total weight of the terpolymer. Another embodiment of the instant invention for component b) formula (I) is that w is from about 5% to about 30% by weight based on the total weight of the terpolymer.

Another embodiment of the instant invention for component b) formula (I) is that M is derived from at least one monomer of formula (II)

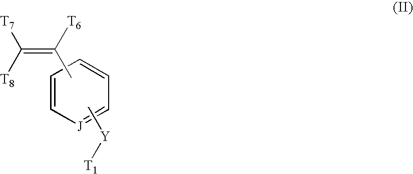

wherein T6, T7, and T8 are methyl, ethyl or hydrogen; Y is a direct bond; T1 is hydrogen or C1-C4 alkyl; and J is a carbon atom.

Another embodiment of the instant invention for component b) formula (I) is that M is derived from at least one monomer of formula (II)

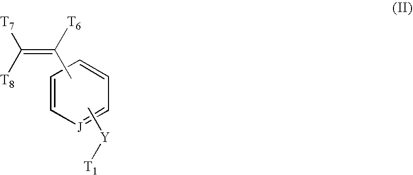

wherein T6, T7, and T8 are methyl or hydrogen; Y is a direct bond; T1 is hydrogen, methyl, or ethyl; and J is a carbon atom.

Another embodiment of the instant invention for component b) formula (I) is that M is derived from at least one monomer selected from the group consisting of styrene, alpha-methylstyrene, 2-vinyltoluene, 3-vinyltoluene, 4-vinyltoluene, ethylvinylbenzene and mixtures thereof.

Another embodiment of the instant invention for component b) formula (I) is T, D, and E are independently derived from at least one monomer of formula (III)

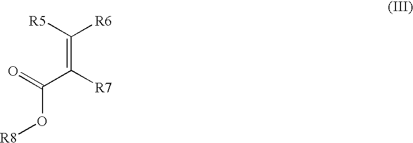

wherein R5, R6 and R7 may be the same or different and represent hydrogen or C1-C12 alkyl;
R8 is C1-C18 alkyl, or 06-015 cycloalkyl; said substituted alkyl, or said cycloalkyl may also be substituted by one or more —OH and/or NH2 groups; said alkyl or said cycloalkyl may be interrupted by one or more —O— groups and/or —N(H)— groups.

Another embodiment of the instant invention for component b) formula (I) is T, D, and E are independently derived from at least one monomer selected from the group consisting of methyl(meth)acrylate, ethyl(meth)acrylate, propyl(meth)acrylate, butyl (meth)acrylate, isobutyl(meth)acrylate, hexyl(meth)acrylate, cyclohexyl(meth)acrylate, 2-ethylhexyl(meth)acrylate, octyl(meth)acrylate, decyl(meth)acrylate, dodecyl (meth)acrylate, dimethyl aminoethyl(meth)acrylate, isobornyl(meth)acrylate, stearyl (meth)acrylate, behenyl(meth)acrylate, polypropylene glycol mono(meth)acrylate, glycidyl(meth)acrylate, polyethylene glycol mono(meth)acrylate, EO-PO-mono(meth)acrylate and mixtures thereof. The parentheses indicate that the monomers of formula (III) are esters based on either methacrylic acid or acrylic acid.

Another embodiment of the instant invention is random terpolymers of component (b) formula (I) that consist of a polymer chain having attached thereto a monomer derived from G containing heterocyclic groups with basic nitrogen atoms. Such a chain can be obtained either by polymerizing-in compounds containing both a vinyl and such a heterocyclic group, or by later attaching a heterocyclic group to the polymer chain containing corresponding reactive groups.

Preferred are heterocyclic groups with basic nitrogen groups having a pKa value of 2 to 14, more in particular 5 to 14 and most preferably 5 to 12. These pKa values relate to the measurement thereof at 25 C in a 0.01 molar concentration in water. These basic groups impart to the random terpolymers according to the invention a basicity. These basic groups allow the random terpolymers to form organic and/or inorganic salts too. The random terpolymers can therefore be used in the form of such salts.

These salts are obtained by neutralization of the polymer with organic acids, e.g., aromatic acids having not more than 25 carbon atoms or aliphatic and cycloaliphatic acids having not more than 22 carbon atoms. Preference is given to salts of the polymer with organic monocarboxylic acids. Inorganic acids are, for example, hydrochloric acid, hydrobromic acid, sulfurous acid, sulfuric acid, and the like.

Suitable compounds of component b formula (I) G to be polymerized-in are selected from the group consisting of vinylimidazole, 2-vinylpyridine, 4-vinylpyridine, 2-methyl-N-vinylimidazole, vinylpyrrolidone, vinylcarbazole and mixtures thereof.

Suitable compounds containing at least one basic nitrogen atom and capable of being attached to a polymer chain of formula (I) G are described in, among others, EP-A 154,678 and U.S. Pat. No. 5,466,749.

Suitable compounds containing at least one basic nitrogen atom and capable of being attached to a polymer chain of formula (I) G are selected from the group consisting of 1-(2-hydroxyethyl)-pyrrolidine, 2-(1-pyrrolidyl)-ethylamine, 2-(1-piperidyl)-ethylamine, 1-(2-hydroxyethyl)-piperidine, 1-(2-aminopropyl)-piperidine, N-(2-hydroxyethyl)-hexamethylenimine, 4-(2-hydroxyethyl)-morpholine, 2-(4-morpholinyl)-ethylamine, 4-(3-aminopropyl)-morpholine, 1-(2-hydroxyethyl)-piperazine, 1-(2-aminoethyl)-piperazine, 1-(2-hydroxyethyl)-2-alkylimidazoline, 1-(3-aminopropyl)-imidazole, (2-aminoethyl)-pyridine, (2-hydroxyethyl)-pyridine, (3-hydroxypropyl)-pyridine, (hydroxymethyl)-pyridine, N-methyl-2-hydroxy-methyl-piperidine, 1-(2-hydroxyethyl)-imidazole, 2-amino-6-methoxybenzothiazole, 4-aminomethyl-pyridine, 4-amino-2-methoxypyrimidine, 2-mercaptopyrimidine, 2-mercapto-benzimidazole, 3-mercapto-1,2,4-triazole, 3-amino-1,2,4-triazole, 2-isopropyl-imidazole, 2-ethyl-imidazole, 4-methyl-imidazole, 2-methyl-imidazole, 2-ethyl-4-methyl-imidazole, 2-phenyl-imidazole, 4-nitro-imidazole and mixtures thereof.

When G contains vinylimidazole, for example, the terpolymer exhibits particularly good adherence to skin.

While not wishing to be bound by theory, it is believed that the vinylimidazole functionality has an affinity for protein. For example, it is known that poly(N-isopropylacrylamide/vinylimidazole) copolymer has an affinity for histidine. See Kumara, K. M. et al, Langmuir, 2003, vol. 19, n° 3, pp. 865-871.

This may explain the exceptional performance of the inventive terpolymers containing heterocyclics such as vinylimidazole and similar heterocycles.

Another embodiment of the instant invention for component b) formula (I) H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bisphenylene diisocyanate, 4,4′-bis(2-methoxyisocyanatophenyl)methane, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, m-tetramethylxylylene diisocyanate and mixtures thereof.

Another embodiment of the instant invention for component b) formula (I) is that H is derived from at least one monomer selected from the group consisting of toluene diisocyanate, 4,4′-diphenylmethane diisocyanate, tolidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bisphenylene diisocyanate, 4,4′-bis(2-methoxyisocyanatophenyl)methane, 4,4′-diisocyanatodiphenyl ether, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, 4,4′-dicyclohexylmethane diisocyanate, isophorone diisocyanate, 3-isocyanatomethyl-3,5,5-trimethylcyclohexyl diisocyanate, lysine methyl ester diisocyanate, m-tetramethylxylylene diisocyanate and mixtures thereof.

The random terpolymers of formula (I) according to the instant invention maybe be crosslinked by multifunctional monomers. These multifunctional monomers are selected from the group consisting of divinyl benzene, trivinylbenzene, divinyltoluene, divinylpyridine, divinylnaphthalene divinylxylene, ethyleneglycol di(meth)acrylate, trimethylolpropane tri(meth)acrylate, diethyleneglycol divinyl ether, trivinylcyclohexane, allyl(meth)acrylate, diethyleneglycol di(meth)acrylate, propyleneglycol di(meth)acrylate, 2,2-dimethylpropane-1,3-di(meth)acrylate, 1,3-butylene glycol di(meth)acrylate, 1,4-butanediol di(meth)acrylate, 1,6-hexanediol di(meth)acrylate, tripropylene glycol di(meth)acrylate, triethylene glycol di(meth)acrylate, tetraethylene glycol di(meth)acrylate, polyethylene glycol di(meth)acrylates, polyethylene glycol 200 di(meth)acrylate, polyethylene glycol 600 di(meth)acrylate, ethoxylated bisphenol A di(meth)acrylate, poly(butanediol) di(meth)acrylate, pentaerythritol tri(meth)acrylate, trimethylolpropane triethoxy tri(meth)acrylate, glyceryl propoxy tri(meth)acrylate, pentaerythritol tetra(meth)acrylate, dipentaerythritol monohydroxypenta(meth)acrylate, divinyl silane, trivinyl silane, dimethyl divinyl silane, divinyl methyl silane, methyl trivinyl silane, diphenyl divinyl silane, divinyl phenyl Mane, trivinyl phenyl silane, divinyl methyl phenyl silane, tetravinyl silane, dimethyl vinyl disiloxane, poly(methyl vinyl siloxane), poly(vinyl hydro siloxane), poly(phenyl vinyl siloxane), and mixtures thereof.

The weight-average molecular weight of the random terpolymer of component (b) formula (I) exhibits a weight-average molecular weight of about 500 Daltons to about 1,000,000 Daltons. In another aspect of the instant invention, the weight-average molecular weight of the random terpolymer of component (b) formula (I) exhibits a weight-average molecular weight of about 500 Daltons to about 500,000 Daltons. In yet another aspect of the instant invention, the weight-average molecular weight of the random terpolymer of component (b) formula (I) exhibits a weight-average molecular weight of about 500 Daltons to about 100,000 Daltons. In still another aspect of the instant invention, the weight-average molecular weight of the random terpolymer of component (b) formula (I) exhibits a weight-average molecular weight of about 1000 Daltons to about 75,000 Daltons or about 1500 to about 50,000 Daltons.

The random terpolymer of component (b) formula (I) is present in the color cosmetic formulation composition in amounts from about 0.01 weight % to about 50 weight % based on the weight of the total composition. In another aspect of the instant invention, the random terpolymer of component (b) formula (I) is present in the sunscreen composition in amounts from about 0.1 weight % to about 25 weight % based on the weight of the total composition. In still another aspect of the instant invention, the random terpolymer of component (b) formula (I) is present in the sunscreen composition in amounts from about 0.1 weight % to about 10 weight % based on the weight of the total composition. For example, an effective amount of coloring agent or pigment will vary depending upon the effect desired in the color cosmetic. Typically the random terpolymer of formula (I) in a color cosmetic formulation will range from about 0.01 to about 10 weight % based on the formulation. For example, about 0.01 to about 5 or 7 weight % is envisioned. More typically, about 0.1 to about 3 or 4 weight % is envisioned.

Another embodiment of the instant invention are random terpolymers of component (b) formula (I) that contain less than 250 ppm of residual monomers. Another embodiment of the instant invention are random terpolymers of component (b) formula (I) that contain less than 200 ppm of residual monomers. Another embodiment of the instant invention are random terpolymers of component (b) formula (I) that contain less than 100 ppm of residual monomers. Another embodiment of the instant invention are random terpolymers of component (b) formula (I) that contain less than 50 ppm of residual monomers. Another embodiment of the instant invention are random terpolymers of component (b) formula (I) that contain less than 5 ppm of residual monomers.

The random terpolymers of formula (I) according to the instant invention are water-dispersible or water soluble and can be distributed throughout the aqueous phase or the oil phase of the instant compositions or formulations.

The random terpolymers of formula (I) are especially good as dispersing agents for organic and inorganic particulates.

The terpolymers are particularly advantageous in reducing oil droplet size in oil-in-water emulsions or water droplet size in water-in-oil emulsions.

Thus the invention embodies a method of reducing the oil droplet size of a cosmetic oil-in-water cosmetic emulsion which method comprises the steps of adding a random terpolymer of formula (I) to the oil, the water phase or to the formed oil-in-water emulsion.

The random terpolymers are also highly compatible with many cosmetic formulations and can be incorporated in the water phase of an emulsion or post-added after the emulsion is formed.

The random terpolymers of the invention form transparent films when no colorant is added. This is a significant advantage in cosmetic formulations because no whitening of the film occurs after drying when the color cosmetic is applied to skin or hair.

Furthermore, no neutralization of the terpolymer is required to effect incorporation into the cosmetic color formulation.

The terpolymer per se in water may be virtually neutral, characterized by a pH ranging from about 5 to 8, more preferably about 5 to about 7.5 and most preferably about 5.2 to about 7.

It is known that increasing salt concentration in a formulation has a tendency to decrease the viscosity of said formulation. The terpolymers of the invention, however, are very tolerant of salt concentrations in a cosmetic formulation and help to maintain formulation viscosity even in the presence of salts.

While not wishing to be bound by theory, it is believed that the multiple advantages of the random terpolymer such as compatibility with diverse cosmetic formulations and ability to disperse particulates such as pigments reside in the multiple functionality of the random terpolymer.

For example, the heterocycle may have an affinity for skin as well as pigment. Thus the terpolymer may anchor to skin and pigment making the color films formed on skin or hair water resistant and smudge and smear resistant. As the terpolymer may additionally contain both hydrophobic and hydrophilic functionality, the terpolymer appears to be compatible with multiple cosmetic formulations. The hydrophobic character of the terpolymer may provide the water resistance while the hydrophilic character of the terpolymer provide water compatibility.

The multifunctionality of the terpolymer also provides amphiphilic characteristics.

### Preparation of Random Terpolymer

The random terpolymers of component (b) formula (I) can be prepared in the conventional manner, e.g., by mass or solution polymerization. The polymerization in a solvent is preferred in view of the controllability of the polymerization and the viscosity of the final product. Suitable solvents are DMSO, THF, DMF, ethyl, propyl, butyl, acetate, benzene, toluene, xylene, N-butanol, isobutanol, isopropanol, MEK, MIBK, acetone, etc.

The monomers are preferably polymerized using a radical reaction, by addition of peroxides, optionally in the presence of redox systems.

The polymerization time of the random terpolymer of component (b) formula (I) depends on the temperature and the desired final product properties but is preferably within the range of from 0.5 to 10 hours at temperatures ranging from about 50 C to about 190 C. The polymerization can be carried out continuously, discontinuously or semicontinuously. If it is preferred to obtain a polymer chain having random distribution of monomers, all of the monomers together will be preferably added to the reaction mixture. This may be done in one portion or in the course of time.

On the basis of the reactivity of the monomers, which is known, a skilled artisan can control the polymerization so as to obtain the desired distribution.

The compositions of the invention may further comprise, cosmetically acceptable ingredients and adjuvants selected, in particular, from among fatty substances, organic solvents, thickeners, demulcents, opacifiers, additional colorants, additional effect pigments, sunscreens, stabilizers, emollients, antifoaming agents, moisturizing agents, antioxidants, vitamins, peptides, amino acids, botanical extracts, particulates, perfumes, preservatives, polymers, fillers, sequestrants, propellants, alkalinizing or acidifying agents or any other ingredient customarily formulated into color cosmetics.

The fatty substances may be an oil or a wax or mixtures thereof, and they also comprise fatty acids, fatty alcohols and esters of fatty acids. The oils may be selected from among animal, vegetable, mineral or synthetic oils and, in particular, from among liquid paraffin, paraffin oil, silicone oils, volatile or otherwise, isoparaffins, polyolefins, fluorinated or perfluorinated oils. Likewise, the waxes may be animal, fossil, vegetable, mineral or synthetic waxes which are also known per se.

Waxes are well known ingredients for mascaras. The waxes are those generally used in cosmetics and dermatology. Mention may be made in particular of beeswax, lanolin wax, Chinese insect waxes, rice wax, carnauba wax, candelilla wax, ouricury wax, cork fiber wax, sugar cane wax, Japan wax, sumach wax, montan wax, microcrystalline waxes, paraffin waxes, ozokerites, ceresin wax, lignite wax, polyethylene waxes and the waxes obtained by Fisher-Tropsch synthesis, and fatty acid esters of glycerides that are solid at 40° C. and better still at more than 55° C. Mention may also be made of the waxes obtained by catalytic hydrogenation of animal or plant oils containing linear or branched C₈-C₃₂ fatty chains. Among these, mention may be made in particular of hydrogenated jojoba oil, hydrogenated sunflower oil, hydrogenated castor oil, hydrogenated coconut oil and hydrogenated lanolin oil.

Mention may also be made of silicone waxes or fluoro waxes.

Exemplary organic solvents include the lower alcohols and polyols.

Of course, one skilled in this art will take care to select this or these optional additional compounds and/or their quantities such that the advantageous properties, in particular the resistance to water, the stability, which are intrinsically associated with the color cosmetic compositions in accordance with the invention are not, or not substantially, altered by the addition(s) envisaged.

The compositions of the invention may be formulated according to techniques well known to this art, in particular those suited for the preparation of emulsions of the oil-in-water or water-in-oil type.

The compositions according to the invention may additionally be formulated for protecting the human epidermis or the hair against the damaging effects of ultraviolet radiation, as an anti-sun composition or as a makeup product.

When the subject compositions are formulated as makeup products for the eyelashes, the eyebrows or the skin, such as a treatment cream for the epidermis, foundation, lipstick, eyeshadow, blusher, mascara or eyeliner, same may be provided in a solid or pasty, anhydrous or aqueous form, such as oil-in-water or water-in-oil emulsions, nonionic vesicular dispersions or alternatively suspensions.

The composition of the instant invention may further comprise a fragrance. The term “perfume” or “fragrance” as used herein refers to odoriferous materials which are able to provide a pleasing fragrance to fabrics, and encompasses conventional materials commonly used in cosmetic compositions to counteract a malodor in such compositions and/or provide a pleasing fragrance thereto. The perfumes are preferably in the liquid state at ambient temperature, although solid perfumes are also useful, particularly cyclodextrin/perfume inclusion complexes for controlled release. Included among the perfumes contemplated for use herein are materials such as aldehydes, ketones, esters and the like which are conventionally employed to impart a pleasing fragrance to liquid and solid personal care or cosmetic compositions. Naturally occurring plant and animal oils are also commonly used as components of perfumes. Accordingly, the perfumes useful for the present invention may have relatively simple compositions or may comprise complex mixtures of natural and synthetic chemical components, all of which are intended to provide a pleasant odor or fragrance when applied to fabrics. The perfumes used in personal care or cosmetic compositions are generally selected to meet the normal requirements of odor, stability, price and commercial availability. The term “fragrance” is often used herein to signify a perfume itself, rather than the aroma imparted by such perfume.

The present invention is directed to a method of improving the water resistance of a color cosmetic on skin or hair which method comprises applying to said skin and/or said hair an effective amount of the color cosmetic composition comprising at least one coloring agent and at least one random terpolymer of formula (I),

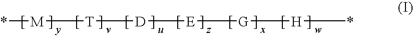

wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (II)

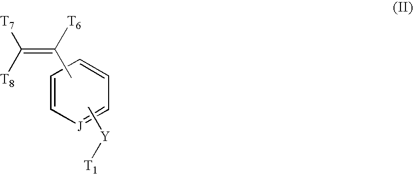

wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III)

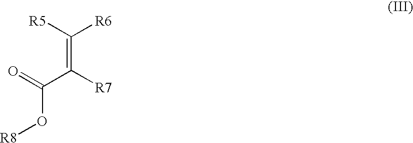

wherein R5, R6 and R7 may be the same or different and represent hydrogen or C1-C22 alkyl;

R8 is C1-C30 alkyl, C6-C15 cycloalkyl, or C6-C15 aryl; said substituted alkyl, said cycloalkyl or said aryl may also be substituted by one or more —OH and/or —NH2 groups; or said alkyl or said cycloalkyl may be interrupted by one or more —O— groups and/or N(H)— groups;

G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile;
with the proviso that T, 0, and E are derived from different monomers
and,
optionally, other cosmetically acceptable ingredients.

The present invention is directed to a topically applicable dermatological, eyebrow or eyelash color cosmetic composition comprising

a) at least one coloring agent
b) a terpolymer of formula (I)

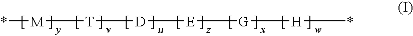

wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
x is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (H)

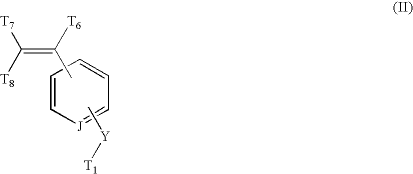

wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III)

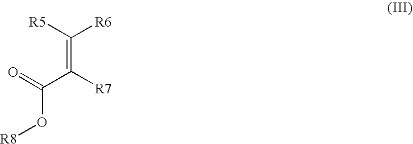

wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more —OH and/or NH2 groups; or said alkyl or said cycloalkyl may be interrupted by one or more —O— groups and/or N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, and acrylonitrile; and
(c) and, optionally other cosmetically acceptable ingredients,
with the proviso that T, D, and E are derived from different monomers.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

Some of the solvents used for the synthesis of the instant copolymers may not be suitable for human physiological conditions. Once the synthesis is completed, the solvents can be removed and/or replaced with solvents that are more cosmetically acceptable.

### Example 1

### Random Terpolymer

In a reaction flask with reflux condenser suitable for polymerization are dissolved in 9.86 g xylene and 4.93 g methoxypropyl acetate 2.84 g vinyl toluene, 4.55 g isobutyl methacrylate, 7.36 g 2-ethylhexyl acrylate, 5.20 g 2-hydroxyethyl methacrylate, 1.80 g polyethylene glycol monomethacrylate having a molecular weight of approximately 400 and 0.44 g ditertiary butyl peroxide. Polymerization is effected at the boiling point of the mixture while stirring and introducing an inert gas. At the end of the polymerization, 9.79 g isophorone diisocyanate are dissolved in 16.58 g isobutyl acetate and 16.58 g methoxypropyl acetate, and the remaining free NCO groups are then converted with 3.60 g polyethylene glycol monomethacrylate having a molecular weight of approximately 400 and 4.51 g 1-(3-aminopropyl)imidazole.

The solid content is then adjusted to 40% by weight with butylacetate,

According to formula (I), component M is vinyl toluene and y is 7.2 weight percent relative to the total weight of the terpolymer; component T is a mixture of isobutyl methacrylate and 2-ethylhexyl acrylate and v is 30.1 weight percent relative to the total weight of the terpolymer; component D is polyethylene glycol monomethacrylate and u is 13.6 weight percent relative to the total weight of the terpolymer; component E is 2-hydroxyethyl methacrylate and z is 13.1 weight percent relative to the total weight of the terpolymer; component G is 1-(3-aminopropyl)imidazole and x is 11.4 weight percent relative to the total weight of the terpolymer; and component H is isophorone diisocyanate and w is 24.7 weight percent relative to the total weight of the terpolymer.

### Example 2

### Random Terpolymer

In the manner comparable with Example 1, 3.54 g vinyl toluene, 5.69 g isobornyl methacrylate, 9.20 g 2-ethylhexyl methacrylate, 7.15 g hydroxy ethyl methacrylate, and 1.28 g ditertiary butylperoxide dissolved in 11.94 g xylene and 5.97 g methoxypropyl acetate are polymerized.

Subsequently, 12.23 g isophorone diisocyanate dissolved in 20.36 g butylacetate and 20.36 g methoxypropyl acetate are added. The remaining free NCO groups are then converted with 4.50 g polyethylene glycol monomethacrylate having a molecular weight of approximate 400 and 3.78 g 3-amino-1,2,4-triazole in 11.34 g N-methylpyrrolidone.

The solid content is then adjusted to 40% by weight with butylacetate.

According to formula (I), component M is vinyl toluene and y is 7.7 weight percent relative to the total weight of the terpolymer; component T is a mixture of isobornyl methacrylate and 2-ethylhexyl methacrylate and v is 32.3 weight percent relative to the total weight of the terpolymer; component D is polyethylene glycol monomethacrylate and u is 9.8 weight percent relative to the total weight of the terpolymer; component E is 2-ethylhexylmethacrylate and z is 15.5 weight percent relative to the total weight of the terpolymer; component G is 3-amino-1,2,4-triazole and x is 8.2 weight percent relative to the total weight of the terpolymer; and component H is isophorone diisocyanate and w is 26.5 weight percent relative to the total weight of the terpolymer.

### Example 3

### Random Terpolymer

In the manner described in Example 1, 6.66 g isobornyl methacrylate, 5.46 g cyclohexyl methacrylate, 6.40 g n-butylacrylate, and 7.85 g 2-hydroxyethyl methacrylate are polymerized with 1.28 g ditertiary butyl peroxide dissolved in 11.98 g xylene and 5.99 g methoxypropyl acetate. To this polymer containing hydroxyl groups, 12.23 g isophorone diisocyanate dissolved in 20.4 g butylacetate and 20.40 g methoxypropyl acetate are added. The free NCO groups are then converted with 4.50 g polyethylene glycol monomethacrylate and 5.54 g 2-(2-pyridyl)-ethanol.

The solid content is then adjusted to 40% by weight with xylene.

According to formula (I), component T is a mixture of isobornyl methacrylate and cyclohexyl methacrylate and v is 24.9 weight percent relative to the total weight of the terpolymer; component D is polyethylene glycol monomethacrylate and u is 9.3 weight percent relative to the total weight of the terpolymer; component E is a mixture of 2-hydroxyethyl methacrylate and butylacrylate and z is 29.3 weight percent relative to the total weight of the terpolymer; component G is 2-(2-pyridyl)-ethanol and x is 11.4 weight percent relative to the total weight of the terpolymer; and component H is isophorone diisocyanate and w is 25.1 weight percent relative to the total weight of the terpolymer.

### Example 4

### Random Terpolymer

In the manner described in Example 1, the polymerization of 3.78 g vinyl toluene, 5.69 g isobutyl methacrylate, 7.38 g 2-ethyl hexyl methacrylate, 7.97 g stearyl methacrylate, 4.55 g glycidyl methacrylate and 0.59 g ditertiary butylperoxide is effected in 14.98 g xylene and 4.99 g methoxypropyl acetate.

At the end of the polymerization, 24.97 g butylacetate and 4.01 g 1-(3-aminopropyl)imidazole are added to the polymer.

According to formula (I), component M is vinyl toluene and y is 11.3 weight percent relative to the total weight of the terpolymer; component T is a mixture of isobutyl methacrylate and 2-ethylhexyl methacrylate and v is 39.1 weight percent relative to the total weight of the terpolymer; component D is stearyl methacrylate and u is 23.9 weight percent relative to the total weight of the terpolymer; component E is glycidyl methacrylate and z is 13.6 weight percent relative to the total weight of the terpolymer; and component G is 1-(3-aminopropyl)imidazole and x is 12.0 weight percent relative to the total weight of the terpolymer.

### Example 5

### Random Terpolymer

In the manner described in Example 1, the polymerization of 6.66 g isobornyl methacrylate, 5.46 g cyclohexyl methacrylate, 9.96 g stearyl methacrylate, 9.22 g 2-ethyl hexyl methacrylate, 5.69 g glycidyl methacrylate and 0.74 g ditertiary butylperoxide is effected in 18.86 g xylene and 6.29 g methoxypropyl acetate.

At the end of the polymerization, 18.94 g butyl acetate and 4.05 g 3-mercapto-1,2,4-triazole dissolved in 16.20 g N-methylpyrrolidone are added to the polymer.

According to formula (I), component T is a mixture of isobornyl methacrylate and cyclohexyl methacrylate and v is 29.5 weight percent relative to the total weight of the terpolymer; component D is a mixture of 2-ethylhexyl methacrylate and stearyl methacrylate and u is 46.8 weight percent relative to the total weight of the terpolymer; component E is glycidyl methacrylate and z is 13.9 weight percent relative to the total weight of the terpolymer; and component G is 3-mercapto-1,2,4-triazole and x is 9.9 weight percent relative to the total weight of the terpolymer.

### Example 6

### Random Terpolymer

In the manner described in Example 1, the polymerization of 12.0 g methyl methacrylate, 32.76 g cyclohexyl methacrylate, 35.84 g butylacrylate, 18.82 g vinyl imidazole and 2.0 g tertiary butyl perbenzoate is effected in 50.71 g xylene and 16.91 g n-butanol.

The solid content is adjusted to 40% by weight with butyl acetate.

According to formula (I), component T is methyl methacrylate and v is 12.1 weight percent relative to the total weight of the terpolymer; component D is cyclohexyl methacrylate and u is 33.0 weight percent relative to the total weight of the terpolymer; component E is butyl acrylate and z is 36.0 weight percent relative to the total weight of the terpolymer; and component G is vinyl imidazole and x is 18.9 weight percent relative to the total weight of the terpolymer.

### Example 7

### Random Terpolymer

In 15.67 g secondary butanol and 47.0 g butyl acetate are polymerized, in the manner described in Example 1: 29.97 g isobornyl methacrylate, 9.36 g styrene, 38.71 g 2-ethyl hexyl acrylate, 14.12 g vinyl imidazole, 0.62 g tertiary butyl-per-2-ethyl hexoate and 1.23 g tertiary butyl perbenzoate.

At the end of the polymerization, the solid content is adjusted to 50% by weight with butyl acetate.

According to formula (I), component M is styrene and y is 10.2 weight percent relative to the total weight of the terpolymer; component T is isobornyl methacrylate and v is 32.5 weight percent relative to the total weight of the terpolymer; component D is 2-ethylhexyl acrylate and u is 42.0 weight percent relative to the total weight of the terpolymer; and component G is vinyl imidazole and x is 15.3 weight percent relative to the total weight of the terpolymer.

### Example 8

### Random Terpolymer

In 45.08 g xylene and 22.54 g n-butanol, 12.00 g methyl methacrylate, 32.76 g cyclohexyl methacrylate, 35.84 g butyl acrylate, 18.82 g vinyl imidazole and 2.0 g tertiary butyl perbenzoate are polymerized in the manner described in Example 1.

At the end of the polymerization, the solid content is adjusted to 50% by weight by adding 33.80 g xylene.

According to formula (I), component T is methyl methacrylate and v is 12.1 weight percent relative to the total weight of the terpolymer; component D is cyclohexyl methacrylate and u is 33.0 weight percent relative to the total weight of the terpolymer; component E is butyl methacrylate and z is 36.0 weight percent relative to the total weight of the terpolymer; and component G is vinyl imidazole and x is 18.9 weight percent relative to the total weight of the terpolymer.

### Example 9

### Random Terpolymer

In 47.00 g toluene and 15.67 g n-butanol, 29.97 g isobornyl methacrylate, 9.36 g styrene, 38.71 g 2-ethyl hexyl acrylate, 14.12 g vinyl imidazole and 11.85 g tertiary butyl perbenzoate are polymerized in the manner described in Example 1.

At the end of the polymerization, a polymer solution is obtained having a solid content of 60% by weight.

According to formula (I), component M is styrene and y is 10.2 weight percent relative to the total weight of the terpolymer; component T is isobornyl methacrylate and v is 32.5 weight percent relative to the total weight of the terpolymer; component D is 2-ethylhexyl acrylate and u is 42.0 weight percent relative to the total weight of the terpolymer; and component G is vinyl imidazole and x is 15.3 weight percent relative to the total weight of the terpolymer.

### Example 10

### Random Terpolymer

In 43.75 g xylene and 14.59 g n-butanol, 23.31 g isobornyl methacrylate, 31.35 g butyl acrylate, 10.92 g styrene, 3.71 g acrylonitrile, 16.47 g vinyl imidazole and 1.72 g tertiary butyl perbenzoate are polymerized.

At the end of the polymerization, the solid content of the polymer solution is adjusted to 50% by weight by adding xylene.

According to formula (I), component M is styrene and y is 12.7 weight percent relative to the total weight of the terpolymer; component T is isobornyl methacrylate and v is 27.2 weight percent relative to the total weight of the terpolymer; component D is butylacrylate and u is 36.6 weight percent relative to the total weight of the terpolymer; component G is vinyl imidazole and x is 19.2 weight percent relative to the total weight of the terpolymer; and H is acrylonitrile and w is 4.3 weight percent relative to the total weight of the terpolymer.

### Example 11

### Random Terpolymer

In the manner described in Example 1, 19.98 g isobornyl methacrylate, 10.62 g vinyl toluene, 30.42 g 2-ethylhexyl acrylate, 6.75 g polyethylene glycol monomethacrylate, 16.38 g cyclohexyl methacrylate, 15.53 g vinyl imidazole, 0.67 g tertiary butyl peroctoate and 1.34 g tertiary butyl perbenzoate are polymerized in 50.85 g butyl acetate and 16.95 g secondary butanol.

At the end of the polymerization, the solid content of the polymer solution is adjusted to 50% by weight with butyl acetate.

According to formula (I), component M is vinyl toluene and y is 10.7 weight percent relative to the total weight of the terpolymer; component T is a mixture of isobornyl methacrylate and 2-ethylhexyl acrylate and v is 50.5 weight percent relative to the total weight of the terpolymer; component D is polyethylene glycol monomethacrylate and u is 6.8 weight percent relative to the total weight of the terpolymer; component E is cyclohexyl methacrylate and z is 16.4 weight percent relative to the total weight of the terpolymer; and component G is vinyl imidazole and x is 15.6 weight percent relative to the total weight of the terpolymer.

### Example 12

### Random Terpolymer

In 98.67 g butyl acetate and 19.74 g n-butanol, the following substances are polymerized, in the manner described in Example 1: 19.98 g isobornyl methacrylate, 10.92 g cyclohexyl methacrylate, 10.62 g vinyl toluene, 15.0 g methyl methacrylate, 6.75 g polyethylene glycol monomethacrylate, 14.12 g vinyl imidazole and 1.56 g tertiary butyl perbenzoate.

At the end of the polymerization, the solid content of the solution is adjusted to 40% by weight by adding butyl acetate.

According to formula (I), component M is vinyl toluene and y is 13.7 weight percent relative to the total weight of the terpolymer; component T is a mixture of isobornyl methacrylate and cyclohexyl methacrylate and v is 39.9 weight percent relative to the total weight of the terpolymer; component D is polyethylene glycol monomethacrylate and u is 8.7 weight percent relative to the total weight of the terpolymer; component E is methyl methacrylate and z is 19.4 weight percent relative to the total weight of the terpolymer; and component G is vinyl imidazole and x is 18.2 weight percent relative to the total weight of the terpolymer.

### Example 13

### Random Terpolymer

The following substances are polymerized randomly, similar to the manner described in Example 11 except sec-butanol is used as the solvent: 9.0 g vinyltoluene, 6.6 g 2-hydroxyethyl methacrylate, 13.2 g vinyl imidazole, 14.1 g 2-ethylhexylacrylate, and 66.9 g monomethoxypolyethylene glycol monomethacrylate. After completion of the polymerization reaction, all solvents and volatiles are removed by vacuum distillation. A polymeric melt is obtained with a molecular weight of about 15,000-20,000 Dalton as determined by Gel Permeation Chromotography (GPC).

According to formula (I), component M is vinyl toluene and y is 8.2 weight percent relative to the total weight of the terpolymer; component T is 2-ethylhexyl methacrylate and v is 12.8 weight percent relative to the total weight of the terpolymer; component D is polyethylene glycol monomethacrylate and u is 60.9 weight percent relative to the total weight of the terpolymer; component E is 2-hydroxyethyl methacrylate and z is 6.0 weight percent relative to the total weight of the terpolymer; and component G is vinyl imidazole and x is 12.0 weight percent relative to the total weight of the terpolymer.

A 50% (w/w) solution of the random terpolymer is prepared by dissolving 109.8 g of the random terpolymer synthesized above in a 109.8 g of Water.

This solution is an easy-to-handle form of the random terpolymer that is suitable for intended applications.

pH of terpolymer at 50% concentration in water is 5.6-6.0

Average Molecular weight of terpolymer of example 13: ˜15,000 to 20,000 Daltons.

Tg of example 13 terpolymer is 2.85° C.

Minimum Film Forming Temperature of example 13 terpolymer is more than 90° C.

### Application Examples

### Example 14

TABLE I
Preparation of mascara emulsions.

| | | | | % w/w | % w/w B | | |
|---|---|---|---|---|---|---|---|
| | | | | A- | No | | |
| Phase | Trade Name | INCI-Name | Supplier | Control | Polymers | % w/w C | % w/w D |
| A | Deionized | Deionized Water | Deionized | 57.30 | 61.30 | 57.30 | 59.30 |
| | Water | | Water | | | | |
| | Butylene | Butylene Glycol | Jeen | 4.00 | 4.00 | 4.00 | 4.00 |
| | Glycol | | International | | | | |
| | | | Corporation | | | | |
| | Versene NA | Disodium EDTA | Dow | 0.10 | 0.10 | 0.10 | 0.10 |
| | | | Chemical | | | | |
| | | | Company | | | | |
| | Example 13¹ | Random | Ciba | — | — | 2.00 | 2.00 |
| | | Terpolymer | | | | (1% | (1% |
| | | | | | | active) | active) |
| B | Natrosol 250 | Hydroxyethylcellulose | Aqualon | 1.00 | 1.00 | 1.00 | 1.00 |
| | HR | | | | | | |
| C | Orgasol | Nylon-12 | Lipo | 1.00 | 1.00 | 1.00 | 1.00 |
| | 2002D Ex | | Chemicals, | | | | |
| | Nat Cos | | Inc. | | | | |
| | Calisha Jet | Black Iron Oxide | Ciba | 10.00 | 10.00 | 10.00 | 10.00 |
| | Black Iron | | | | | | |
| | Oxide | | | | | | |
| D | White | Beeswax | Frank B. | 10.00 | 10.00 | 10.00 | 10.00 |
| | Bleached | | Ross | | | | |
| | Beeswax | | | | | | |
| | Copernica | Carnauba Wax #1 | Frank B. | 6.60 | 6.60 | 6.60 | 6.60 |
| | Cerifera | Yellow | Ross | | | | |
| | (carnauba | | | | | | |
| | wax) | | | | | | |
| | VP/Eicosene | Ganex V-220 | ISP | 2.00 | — | 2.00 | — |
| | Copolymer | | | | | | |
| | PEG-20 | Cerasynt 840 | ISP | 2.00 | 2.00 | 2.00 | 2.00 |
| | Stearate | | | | | | |
| | Glyceryl | Cerasynt 945 | ISP | 3.00 | 3.00 | 3.00 | 3.00 |
| | Stearate (and) | | | | | | |
| | Laureth-23 | | | | | | |
| E | Allianz OPT | Acrylates/C12-22 | ISP | 2.00 | — | — | — |
| | | Alkylmethacrylate | | | | | |
| | | Copolymer | | | | | |
| F | Germaben II | Propylene Glycol | ISP | 1.00 | 1.00 | 1.00 | 1.00 |
| | | (and) Diazolidinyl | | | | | |
| | | Urea (and) | | | | | |
| | | Methylparaben (and) | | | | | |
| | | Propylparaben | | | | | |

¹The example 13 terpolymer is a 50 wt. % solution in water. Thus the concentration of the polymer of the invention is 1 wt. % based on active.

### Procedure:

Heat Water to 82-85 C. Add remaining Phase A ingredients: mix until uniform. Sprinkle Natrosol into batch using slow homo-mixing. Avoid aeration. Add Phase C color phase (Pre-pulverized) to main batch; mix until uniform using homo-mixing. Heat Phase D to 87 C. Add Phase D to main batch using homo-mixing. Mix until uniform. Add Phase E to batch using homo-mixing. Mix until uniform. Switch to sweep-mixing; cool batch to 45 C. Add Phase F to batch; mix until uniform. Continue sweep-mixing and slow cooling to 30° C. Fill in appropriate containers.

### Results

Addition of random terpolymer in Phase A for formulations C and D above gives better dispersion of the pigment/iron oxide and improves water resistant properties of the mascara.

### Example 15

TABLE II
Liquid Foundation
Composition

| | | | | % w/w |
|---|---|---|---|---|
| | | | | (as |
| | Trade Name | INCI-Name | Supplier | supplied) |
| Phase A | Jeecol CS-20 | Ceteareth-20 | Jeen | 4.00 |
| | | | International | |
| | | | Corporation | |
| | Cetiol OE | Dicaprylyl Ether | Cognis | 5.00 |
| | | | Corporation | |
| | Rita OP | Ethylhexyl Palmitate | Rita Corporation | 5.00 |
| | Rita OMC | Ethylhexyl Methoxycinnamate | Rita Corporation | 3.00 |
| | Jeelan MOD | Acetylated Lanolin Alcohol | Jeen | 5.00 |
| | | | International | |
| | | | Corporation | |
| | Abil WE 09 | Polyglycerol-4 Isostearate | Evonik | 4.00 |
| | | (and) Cetyl PEG/PPG-10/1 | Industries | |
| | | Dimethicone (and) Hexyl | | |
| | | Laurate | | |
| | Dow Corning 200 fluid | Dimethicone | Dow Corning | 3.00 |
| | (50 cst) | | Corporation | |
| | Neo Heliopan BB | Benzophenone-3 | Symrise | 5.00 |
| | Tinogard ™ TT DD | Pentaerythrityl Tetra-di-t-butyl | Ciba | 0.05 |
| | | Hydroxyhydrocinnamate | | |
| Phase B | Deionized Water | Deionized Water | Internal | QS to 100 |
| | Propylene Glycol USP | Propylene Glycol | Protameen | 5.00 |
| | | | Chemicals, Inc. | |
| | Random Terpolymer | Random Terpolymer | Ciba | 0.50-2.00 |
| | | | | (.25-1.00% |
| | | | | active) |
| Phase C | Tipaque TiO2-PF671 | Titanium Dioxide | Tipaque | 6.00 |
| | CALISHA ™ Sun Yellow | Iron Oxide | Ciba | 1.08 |
| | Iron Oxide | | | |
| | CALISHA ™ Medium Red | Iron Oxide | Ciba | 0.55 |
| | Iron Oxide | | | |
| | CALISHA ™ Jet Black Iron | Iron Oxide | Ciba | 0.10 |
| | Oxide | | | |
| Phase D | Dow Corning 345 Fluid | Cyclopentasiloxane (and) | Dow Corning | 2.00 |
| | | Cyclohexasiloxane | Corporation | |
| Phase E | Germall Plus Liquid | Propylene Glycol (and) | Sutton | 0.75 |
| | | Diazolidinyl Urea (and) | Laboratories | |
| | | Iodopropynyl Butylcarbamate | | |

### Procedure:

Heat Phase A to 75° C.-80° C. and mix until uniform.
Heat Phase B to 70° C.-75° C. and slowly add gums under high speed propeller agitation.
Mix Phase B until gums are fully hydrated and uniform.
Premix Phase C pigments in an Osterizer/blender until pigments are uniform and slowly add into Phase B under high speed homogenization. Maintaining temperature at (70° C.-75° C.)
Add Phase A to B under high speed homogenization for 15 minutes; Maintaining temperature at (70° C.-75° C.)
Begin to cool batch to 40° C. with sweep or propeller mixing.
Below 40° C. add Phases D, and mix until uniform.

### Example 17

TABLE III
Liquid Eyeliner Formulations
Composition

| | | | | % w/w |
|---|---|---|---|---|
| | | | | (as |
| | Trade Name | INCI-Name | Supplier | supplied) |
| Phase A | Deionized Water | Water | Internal | QS to 100 |
| | Versene Na | Disodium EDTA | Dow Chemical | 0.10 |
| | | | Co. | |
| Phase B | Natrosol 250 MBR CS | Hydroxyethylcellulose | Hercules | 0.60 |
| Phase C | CALISHA ™ | Ultramarines | Ciba | 3.00 |
| | Contemporary | | | |
| | Ultramarine Blue | | | |
| | CALISHA ™ | Iron Oxide | Ciba | 7.00 |
| | Contemporary Jet Black | | | |
| Phase D | Deionized Water | Water | Internal | 7.50 |
| | Random Terpolymer | Random Terpolymer | Ciba | 0.50-2.00 |
| | | | | (.25-1.00 |
| | | | | active) |
| Phase E | White Beeswax | Beeswax | Strahl & Pitsch, | 5.00 |
| | | | Inc. | |
| | Carnauba Wax #1 | Carnauba Wax | Strahl & Pitsch, | 3.30 |
| | | | Inc. | |
| | Jeenate 1000-DPS | PEG-20 Stearate | Jeen Corporation | 2.00 |
| | Cerasynt 945 | Glyceryl Stearate (and) | International | 3.00 |
| | | Laureth-23 | Specialty | |
| | | | Products, Inc. | |
| Phase F | Germall Plus | Diazolidinyl Urea (and) | International | 0.20 |
| | | Iodopropynyl Butylcarbamate | Specialty | |
| | | | Products, Inc. | |
| Phase G | CALISHA ™ Art Deco | Mica (and) Titanium Dioxide | Ciba | 10.00 |
| | Silver Pearl OD | (and) hydrophobic treatment | | |

### Procedure:

Heat Phase A to 85° C. and mix until uniform
Slowly sprinkle in Natrosol and mix until uniform using homogenizer (Maintain temperature)
Add Phase C colorants (pre-blended) under homogenization and mix until uniform
Premix Phase D at room temperature and add to main vessel. Mix until uniform at 85° C.
Heat Phase E to 87° C. Add to main vessel and mix until uniform
Switch to slow sweep mixing and begin cooling to 45° C.
Add Phase F and Phase G respectively
Continue sweep mixing and slowly cool to 30° C.

### Example 18

### Testing Protocol

The test protocol described below is used to mimic the application of the cosmetic composition onto human skin. This is done by using a UV absorber as a marker. Thus the initial SPF and the SPF after eighty minutes of water exposure are compared. The amount of residual SPF remaining after water exposure is a measure of the water resistance of the film formed from the cosmetic composition onto human skin.

The following laboratory equipment is used:

### Materials and Equipment:

VITRO-SKIN® N-19, Foam block, Hydration Chamber, Powder Free Rubber Finger Cots and Glassless slide mounts are obtained from IMS, Inc. (70 Robinson Blvd, Orange, Conn. 06477, Tel. 203-795-9047).
Water bath (# 05-719-7F), Corning Hotplate Stirrer (#1′-497-8A), Calfamo Compact
Digital Stirrer (#14-500-7), Glycerol Aqueous Solution (#AC277366-0010) are obtained from Fisher Scientific Catalog.
Optometrics SPF 290 was obtained from Optometrics LLC. (8 Nemco Way, Stony Brook Industrial Park, Ayer, Mass. 01432, Tel. 978-772-1700).
Analytical balance—Mettler Toledo AB204-S
Pre-Hydration Step: 300 g of 14.7% aqueous solution of Glycerin is prepared and poured on the bottom of the Hydration chamber. The shelves are placed in the chamber that is covered with the lid. VITRO-SKIN substrate is cut into 4.1 cm×4.1 cm pieces that are put on the shelves in a Hydration chamber for 16-22 hours prior to the tests.

instrument Calibration: Optometrics SPF 290S is turned on followed by the manufacturer's directions for instrument calibration, blank and sample measurements.

Product Application: A piece of substrate is placed in a slide mount and used as a reference for the in vitro SPF measurements. Another piece of substrate is placed on a plastic-covered foam block and product application is made to the “topography” side of the substrate (the rough side). Exactly 0.033 g of product is applied evenly across a 4 cm×4 cm section of the substrate, which resulted in an application dose of 2 mg/sq. cm and rubbed into the substrate with a finger covered with finger cot. After that the substrate is placed in a slide mount.

### Measurements:

1. The in vitro SPF measurements are made both prior to and after sample immersion in water with stirring for 80 minutes. All initial measurements are made after the 15 minute dry-down period. After water exposure the samples are removed, air-dried for about 30 minutes, placed back in the controlled humidity chamber for 120 minutes followed by the minute dry-down period. The reference slides are immersed in the water bath for the same amount of time.

An Optometrics SPF 290S is used to determine UV absorbance for each formulation in the 290-400 nm wavelength range, A minimum of three consecutive measurements on three separate areas of the slide are conducted. SPF, UVA/UVB and Critical Wavelength in vitro values for each sample—before and after water immersion are recorded. The % SPF remaining after eighty minute exposure to water is calculated by:

(a/b)×100=% SPF remaining

(a) is SPF value after 80 minutes of water exposure and (b) is initial SPF value.
2. Alternatively, organic and inorganic pigments and effect pigments can be utilized as markers. Color measurements of the samples before and after water exposure are conducted. A COLORTEC PSM; Color Mode: L*a*b*; Observer 10°; Primary Illuminant D65 (Color-Tec, Clinton—N.J.) is utilized. All colorimetric measurements are conducted in the CIE (1976) L*a*b color space. Delta E*ab values, a mathematical description of the distance between two colors that provides a measurement of both hue and density changes are utilized to evaluate the color change of samples after water exposure.

dE*ab=\sqrt{[(dL*²)+(da*²)+(db*²)]}

### Example 19

### Testing for Water Resistant Properties

TABLE V
Cosmetic Emulsions Containing Oil-soluble and Organic Particulate Sunscreens as
Markers to Determine Water Resistance of Terpolymer

| Part | Trade name | INCI name | Function | % W/W* |
|---|---|---|---|---|
| A | Arlacel 165 | Glyceryl Stearate (and) | Emulsifier, non-ionic | 3.50 |
| | | PEG-100 Stearate | | |
| | Lanette 16 | Cetyl Alcohol | Emulsion Stabilizer | 1.00 |
| | Cetiol B | Dibutyl Adipate | Emollient/solvent | 5.00 |
| | Cetiol CC | Dicaprylyl Carbonate | Emollient/solvent | 5.00 |
| | Tegosoft DEC | Diethylhexyl Carbonate | Emollient/solvent | 2.00 |
| | Neo Heliopan AV | Octinoxate | Sunscreen Active | 7.50 |
| | Neo Heliopan OS | Octisalate | Sunscreen Active | 2.00 |
| | Parsol 1789 | Avobenzone | Sunscreen Active | 3.00 |
| | Tinosorb ® S | Drug name (proposed): | Sunscreen Active | 1.50 |
| | | Bemotrizinol; | | |
| | | INCI name: Bis- | | |
| | | Ethylhexyloxyphenol | | |
| | | Methoxyphenyl Triazine | | |
| | Orgasol 2002 EXD | Nylon-12 | Spherical Particulate to | 1.50 |
| | NAT COS | | improve skin feel | |
| B | Water | Water | Diluent/solvent | Qs to 100.00 |
| | Keltrol CG RD | Xanthan Gum | Rheology Modifier | 0.22 |
| | Glycerin | Glycerin | Humectant | 3.00 |
| | Dissolvine NA2 | Disodium EDTA | Chelating Agent | 0.20 |
| C | Dow Corning 345 | Cyclopentasiloxane | Emollient/Solvent | 2.00 |
| | Fluid | | | |
| | Tinovis ADM | Sodium Acrylates | Rheology Modifier | 0.83 |
| | | Copolymer (and) Mineral | | |
| | | Oil (and) PPG-1 Trideceth-6 | | |
| | Tinosorb ® M | Drug name (proposed): | Sunscreen Active | 3.00 (as is) |
| | (insoluble org. | Bisoctrizole; | | or |
| | particulate, see co- | INCI name: Methylene Bis- | | 1.50 (active |
| | pending 12/079,607) | Benzotriazolyl | | level) |
| | | Tetramethylbutylphenol | | |
| | Phenonip | Phenoxyethanol (and) | Preservative | 1.00 |
| | | Methylparaben (and) | | |
| | | Ethylparaben (and) | | |
| | | Butylparaben (and) | | |
| | | Propylparaben (and) | | |
| | | Isobutylparaben | | |
| | Sodium Hydroxide | Sodium Hydroxide (15% | pH adjustor | Qs to pH ~5.3-6.1 |
| | | aqueous solution) | | |

*The components are added to the sunscreen composition on a % weight/weight of component (as active) based on the weight of the total composition.

Combine the ingredients of part A. Heat up part A to 80° C. with mixing. Mix until uniform, and add Nylon-12 with moderate agitation.

Prepare part B: first, disperse Xanthan Gum into the water and heat up to 80° C. When uniform, add the rest of part B one by one, mix until uniform.

Add part A into part B under stirring, and then homogenize with an Ultra Turrax pos 2 for 40 sec/100 g.

Cool down under stirring, to 40° C. and add the ingredients of part C one by one in the given order. Mix until uniform. If necessary, adjust pH with aqueous solution of sodium sydroxide to 5.3-6.1

The terpolymer of Instant Example 13 is compared with other commercially available polymers and copolymers. The composition of Table V is prepared individually with the specified amount of each test polymer or copolymer. Commercially available polymers are added to the oil phase or water phase of the formulation, or post-added according to the recommendations described in the manufacturer's literature.

Each color cosmetic formulation is evaluated according to the testing protocol of Example 18.

The data demonstrate the terpolymer provides excellent water proofing properties in cosmetic compositions when compared to other polymers and copolymers of the prior art and commerce.

### Example 20

### Composition Testing for Water Resistant Properties

The base sunscreen composition of Table V formulated with the terpolymer of Instant Example 13 and compared with other commercially available polymers and copolymers. The composition of Table V is prepared individually with the specified amount of each test polymer or copolymer. Commercially available polymers are added to the oil phase or water phase of the formulation, or post-added according to the recommendations described in the manufacturer's literature.

Each sunscreen formulation is evaluated according to the testing protocol of Example 18. The experimental results are given below in Table VI.

TABLE VI

| Polymer Tested | wt/wt %* | % SPF remaining |
|---|---|---|
| None | 0 | 3.1 |
| Cosmedia DC | 3 | 4.5 |
| Polycrylene | 3 | 5.7 |
| DC FA 4001 CM | 3 | 6.0 |
| Silicone Acrylate | | |
| Ganex V-220 | 3 | 6.8 |
| DC FA 4002 ID | 3 | 7.4 |
| Silicone Acrylate | | |
| Phospholipon 90H | 3 | 35.0 |
| Dermacryl AQF | 3 | 40.5 |
| Ganex WP-660 | 3 | 53.0 |
| Stantiv OMA-2 | 3 | 53.3 |
| Dermacryl-79 | 3 | 55.9 |
| Allianz OPT | 3 | 81.1 |
| Avalure UR 450 | 3 | 83.5 |
| Instant Example 13 | 1 | 87.4 |

*The commercially available polymers are added to the sunscreen composition at a 3% weight/weight of component (as active) based on the weight of the total composition.

Example 13 is added at a 1% weight/weight of component (as active) based on the weight of the total composition.

Cosmedia DC is a hydrogenated dimer Dilinoleyl/Dimethylcarbonate Copolymer and is obtained from Cognis.

Polycrylene is Polyester-8 which is a copolymer of adipic acid (q.v.) and neopentyl glycol (q.v.) end-capped with either octyldodecanol (q.v.) or a cyanodiphenylpropenoyl group and is obtained from RTD Hall Star.

DC FA 4001 CM Silicone Acrylate is a copolymer of polytrimethylsiloxymethacrylate and one or more monomers consisting of acrylic acid, methacrylic acid, or one of their simple esters dissolved in cyclopentasiloxane and is obtained from Dow Corning.

Ganex V-220 is a copolymer of vinylpyrrolidone and eicosene and is obtained from ISP.

DC FA 4002 ID Silicone Acrylate is a copolymer of polytrimethylsiloxymethacrylate and one or more monomers consisting of acrylic acid, methacrylic acid, or one of their simple esters dissolved in isododecane and is obtained from Dow Corning.

Phospholipon 90H is hydrogenated lecithin and is obtained from Phospholipid GmbH.

Dermacryl AQF is a copolymer of acrylates and is obtained from National Starch and Chemical Company.

Ganex WP-660 is a copolymer of vinyl pyrrolidone and 1-triacontane and is obtained from ISP.

Stantiv OMA-2 is a linear copolymer of maleic anhydride and octadecene and is dissolved a mixture of methyl acetyl ricinoleate and dimethylheptyl adipate.

Dermacryl-79 is a copolymer of octylacrylamide and one or more monomers consisting of acrylic acid, methacrylic acid or one of their simple esters and is obtained from National Starch and Chemical Company.

Allianz OPT is a copolymer of: methacrylic acid, methyl methacrylate, butyl acrylate, and cetyl-eicosinyl methacrylate and is obtained from ISP.

Avalure UR 450 is a copolymer of PPG-17, isophorone diisocyanate and dimethylol propionic acid monomers and is obtained from Noveon.

The data demonstrate the terpolymer of the invention provides excellent water proofing properties in cosmetic compositions at one-third of the concentration when compared to other polymers and copolymers of the prior art and commerce.

### Example 21

### Sunscreen Composition Testing for Water Resistant Properties

A commercial sunscreen formulation (Cetaphil SPF 15, Galderma) is obtained and is thoroughly mixed individually with the specified amount of each test polymer or copolymer. Each sunscreen formulation is evaluated according to the testing protocol of Example 18. The experimental results are given below in Table VII.

TABLE VII

| Polymer Tested | wt/wt %* | % SPF remaining |
|---|---|---|
| None | 0 | 10.1 |
| Dermacryl AQF | 2 | 9.1 |
| Allianz OPT | 1 | 49.1 |
| Instant Example 13 | 1 | 62.9 |

*The polymers are added to the sunscreen composition on a % weight/weight of component (as active) based on the weight of the total composition.

Dermacryl AQF is a copolymer of acrylates and is obtained from National Starch and Chemical Company.

Allianz OPT is a copolymer of: methacrylic acid, methyl methacrylate, butyl acrylate, and cetyl-eicosinyl methacrylate and is obtained from ISP.

Cetaphil SPF 15 is a commercial sunscreen formulation that contains sunscreen actives: Avobenzone 3%; Octocrylene 10%; and

### Inactive Ingredients (Function):

Water (solvent),
Isopropyl adipate (emollient, solvent),
Cyclomethicone (emollient, solvent),
Glyceryl Stearate (and) PEG-100 Stearate (emulsifier, non-ionic),
Glycerin (humectant),
Polymethyl Metacrylate (spherical particulate to improve the skin feel,
Phenoxyethanol (preservative),
Benzyl Alcohol (preservative),
Acrylates/C10-30 Alkyl Acrylate Crosspolymer (polymeric emulsifier, rheology modifier),
Tocopheryl Acetate (antioxidant),
Carbomer (rheology modifier),
Disodium EDTA (chelating agent), and
Triethanolamine (pH adjustor).

The data demonstrate the instant terpolymer provide excellent water proofing properties in compositions when compared to other polymers and copolymers of the prior art and commerce.

### Example 22

### Contact Angles of Water and Surface Properties

A test methodology that utilizes measurements of the contact angle of water to quantify the effects on the surface properties of a skin-substitute substrate is employed. This methodology is used as an effective tool for optimizing product development, differentiating among skin care products, competitive benchmarking, and screening of the polymers. It is described in the article entitled “Correlating Water Contact Angles and Moisturization/Sensory Claims” by Olga V. Dueva-Koganov, Scott Jaynes, Colleen Rocafort, Shaun Barker and Jianwen Mao—Cosmetics & Toiletries, January 2007, Vol. 122, No. 1, pp. 20-27. The data presented in the graph of this article shows that contact angle measurements can be used to quantify and compare the effects of skin care products on the surface properties of a skin-like substrate and is presented in tabular form below. Products that generate relatively low contact angles tend to make more sensory claims related to light and non-greasy feel, while products that produce relatively high contact angles tend to make more claims related to long-term moisturization.

TABLE VIII

| Contact | | | |
|---|---|---|---|
| Angles | % Products in A* | % Products in B** | % Products in C*** |
| 40-50 | 100 | 50 | 0 |
| 50-60 | 100 | 40 | 20 |
| 60-70 | 60 | 30 | 60 |
| 70-80 | 70 | 15 | 70 |
| 80-90 | 50 | 0 | 100 |

*A = Products that are light and/or nongreasy.
**B = Products that provide 8-12 hours moisturization.
**C = Products that provide 24 hours moisturization.

### Example 23

### Measurement of Contact Angles after Application of the Instant Terpolymers

Contact angles are measured instrumentally according to the static or sessile drop method and using deionized water as a probe solution and VITRO SKIN that mimics the surface properties of human skin as a substrate. A piece of hydrated substrate is mounted in a glassless slide and air-dried in a flat position with application side up for 15 minutes. It is used as a reference for untreated substrate during the contact angle measurements. Exactly 0.032 g of aqueous solutions or dispersions of test polymers are applied evenly across a 4 cm×4 cm section of the substrate (on the “skin topography” side). Immediately after product application, the product is rubbed into the substrate with a finger covered with fingercot. After that the substrate is placed in a slide mount and air-dried for 15 minutes. Before measurements, substrate is removed from the slide mount and cut to several small pieces, which are used for the measurements. The use of small size piece is necessary to assure its flat position on the sample table. Extra care is taken to ensure that the rough side is up and the film is flat. Contact angle measurements are conducted expeditiously—within approximately 1 minute. Controlled humidity conditions are utilized.

### Materials

### DSA-10 Contact Angle Measuring System, KrUss Gmb.

### VITRO SKIN(N-19), IMS Inc.,

Powder Free Rubber Finger Cots (# 11-392-9B) are available from the Fisher Scientific Catalog.

Instant terpolymers and competitive water-resistant polymers Allianz OPT (ISP) and Dermacryl AQF (National Starch) are evaluated according to the methodology described above. Results are given in Table IX

TABLE IX
Contact Angles after Application of the Polymers

| | | Contact Angle, |
|---|---|---|
| Polymer Tested | wt/wt %* | Theta (M) deg. |
| Control (Vitro Skin) | None | 103.6 |
| Instant Example 13 | 5 | 60.8 |
| Instant Example 13 | 2.5 | 62.8 |
| Instant Example 13 | 1 | 62.7 |
| Instant Example 13 | 0.5 | 60.3 |
| Instant Example 13 | 0.25 | 71.3 |
| Allianz OPT | 5 | 109.8 |
| Allianz OPT | 2.5 | 108.6 |
| Allianz OPT | 1 | 111.9 |
| Allianz OPT | 0.5 | 99.7 |
| Allianz OPT | 0.25 | 104.8 |
| Dermacryl AQF | 5 | 116.1 |
| Dermacryl AQF | 2.5 | 117.3 |

*Indicates aqueous solutions of the test sample as percent polymer solids based on the total weight of the solution.

Dermacryl AQF is a copolymer of acrylates and is obtained from National Starch and Chemical Company.

Allianz OPT is a copolymer of methacrylic acid, methyl methacrylate, butyl acrylate, and cetyl-eicosinyl methacrylate and is obtained from ISP,

The instant terpolymers and competitive water-resistant polymers demonstrate strong differences in their effects on the surface properties of VITRO SKIN. The results presented in the table above indicate that the instant terpolymers can potentially contribute to light skin feel—a desirable characteristic for water resistant polymers. On the contrary—the competitive benchmarks (Allianz OPT and Dermacryl AQF) generate primarily a hydrophobic modification of the substrate and are less likely to produce light skin feel.

### Example 24

### Sensory Characteristics of Formulations Containing Instant Terpolymers

TABLE X

| Part | Trade name | INCI name | Function | % W/W* |
|---|---|---|---|---|
| A | Arlacel 165 | Glyceryl Stearate (and) | Emulsifier, non-ionic | 3.50 |
| | | PEG-100 Stearate | | |
| | Lanette 16 | Cetyl Alcohol | Emulsion Stabilizer | 1.00 |
| | Cetiol B | Dibutyl Adipate | Emollient/solvent | 5.00 |
| | Cetiol CC | Dicaprylyl Carbonate | Emollient/solvent | 5.00 |
| | Tegosoft DEC | Diethylhexyl Carbonate | Emollient/solvent | 2.00 |
| | Neo Heliopan AV | Octinoxate | Sunscreen Active | 7.50 |
| | Neo Heliopan OS | Octisalate | Sunscreen Active | 2.00 |
| | Parsol 1789 | Avobenzone | Sunscreen Active | 3.00 |
| | Tinosorb ® S | Drug name (proposed): | Sunscreen Active | 1.50 |
| | | Bemotrizinol; | | |
| | | INCI name: Bis- | | |
| | | Ethylhexyloxyphenol | | |
| | | Methoxyphenyl Triazine | | |
| | Orgasol 2002 EXD | Nylon-12 | Spherical Particulate to | 1.50 |
| | NAT COS | | improve skin feel | |
| B | Water | Water | Diluent/solvent | Qs to 100.00 |
| | Keltrol CG RD | Xanthan Gum | Rheology Modifier | 0.22 |
| | Glycerin | Glycerin | Humectant | 3.00 |
| | Dissolvine NA2 | Disodium EDTA | Chelating Agent | 0.20 |
| C | Dow Corning 345 | Cyclopentasiloxane | Emollient/Solvent | 2.00 |
| | Fluid | | | |
| | Tinovis ® ADM | Sodium Acrylates | Rheology Modifier | 0.83 |
| | | Copolymer (and) Mineral | | |
| | | Oil (and) PPG-1 Trideceth-6 | | |
| | Tinosorb ® M | Drug name (proposed): | Sunscreen Active | 3.00 (as is) |
| | | Bisoctrizole; | | or |
| | | INCI name: Methylene Bis- | | 1.50 (active |
| | | Benzotriazolyl | | level) |
| | | Tetramethylbutylphenol | | |
| | Phenonip | Phenoxyethanol (and) | Preservative | 1.00 |
| | | Methylparaben (and) | | |
| | | Ethylparaben (and) | | |
| | | Butylparaben (and) | | |
| | | Propylparaben (and) | | |
| | | Isobutylparaben | | |
| | Sodium Hydroxide | Sodium Hydroxide (15% | pH adjustor | Qs to pH ~5.3-6.1 |
| | | aqueous solution) | | |

*The components are added to the sunscreen composition on a % weight/weight of component (as active) based on the weight of the total composition.

Combine the ingredients of part A. Heat up part A to 80° C. with mixing. Mix until uniform, and add Nylon-12 with moderate agitation.

Prepare part B: first, disperse Xanthan Gum into the water and heat up to 80° C. When uniform, add the rest of part B one by one, mix until uniform.

Add part A into part B under stirring, and then homogenize with an Ultra Turrax pos 2 for 40 sec/100 g.

Cool down under stirring, to 40° C. and add the ingredients of part C one by one in the given order. Mix until uniform. If necessary, adjust pH with aqueous solution of sodium sydroxide to 5.3-6.1

Formulations are prepared as above and tested for sensory characteristics according to testing protocols published in: 1) ASTM, American Society for Testing and Materials; Annual Book of ASTM Standards, E 1490-92 (reapproved 1997), or 2) Meilgaard M, Civille G, Carr B (2007), Sensory Evaluation Techniques, CRC Press, 4th ed.].

The results are given below.

TABLE XI

| Sensory | | | |
|---|---|---|---|
| Characteristics | Form. A | Form. B | Form. C |
| Spreadability | 2 | 2 | 2 |
| Playtime | 3 | 3 | 3 |
| Softness | 4 | 4 | 4 |
| Shiny | 3 | 3 | 3 |
| Greasy | 2 | 2 | 2 |
| Sticky | 2 | 2 | 2 |

Form. A has no instant terpolymers added.
Form. B contains 2% as supplied or 1 percent by weight (solids) of Example 13.
Form. C contains 4% as supplied or 2 percent by weight (solids) of Example 13.

These data demonstrate that the terpolymers of the instant invention do not negatively impact the sensory parameters of the formulation.

### Example 25

TABLE XII
Oil Droplet Size of Oil-in-Water Emulsions

| | | A | B | C | D |
|---|---|---|---|---|---|
| Phase | Ingredients | % wt. | % wt. | % wt. | % wt. |
| Water Phase | Di Water | 68.20 | 67.70 | 67.20 | 66.20 |
| | Disodium EDTA | 0.10 | 0.10 | 0.10 | 0.10 |
| | Glycerin | 3.00 | 3.00 | 3.00 | 3.00 |
| | Example 13 | 0.00 | 0.50 | 1.00 | 2.00 |
| | terpolymer | | | | |
| | (as supplied) | | | | |
| Oil Phase | C12-15 Alkyl | 2.00 | 2.00 | 2.00 | 2.00 |
| | benzoate | | | | |
| | Homosalate | 10.00 | 10.00 | 10.00 | 10.00 |
| | Octisalate | 5.00 | 5.00 | 5.00 | 5.00 |
| | Octocrylene | 2.35 | 2.35 | 2.35 | 2.35 |
| | Avobenzone | 3.00 | 3.00 | 3.00 | 3.00 |
| | Oxybenzone | 4.50 | 4.50 | 4.50 | 4.50 |
| Preservative Phase | Phenonip XB | 0.75 | 0.75 | 0.75 | 0.75 |
| Rheology | Tinovis ® | 1.10 | 1.10 | 1.10 | 1.10 |
| Modifier | ADE | | | | |
| Phase | | | | | |

| | | | | |
|---|---|---|---|---|
| Total | 100.00 | 100.00 | 100.00 | 100.00 |
| Viscosity, cPs (TC, | 27000 | 434000 | 48600 | 44800 |
| 3 rpm, DV-II = Viscometer | | | | |
| % SPF remaining after | 56 | 91 | 100 | 100 |
| Water Exposure | | | | |
| according to Example | | | | |
| 18 Testing Protocol | | | | |

Procedure: in a main vessel combine water phase ingredients one by one, mixing until uniform with propeller agitation (500 rpm) at room temperature. In a side vessel combine oil phase ingredients, heat to 40-45° C. Mix until uniform. Add oil phase to water phase, mix at 1000 rpm (propeller) for 10 min. Add preservative phase. Mix for additional 5 min. (1000 rpm, propeller). Add rheology modifier phase ingredient, Tinovis® ADE, mix for 5 min. with propeller agitation at 2000 rpm. Homogenize for 1 min.

Micrographs of the emulsions were obtained and analyzed on the Hirox HiScope System, Model KH-3000 (Hi Scope System Company, 10 McKinley Street, Suite #12, Closter, N.J.) and oil droplet sizes of the o/w emulsions were determined. Hirox HiScope System, Model KH-3000 includes: Main Control Unit with Digital CCD Camera; High Resolution Digital Color CCD Camera Unit. (2.1M Pixels); Remote CCD Camera Head (⅔″ chip); Fiber optic cable for internal illumination; Internal light source (Metal Halide Arc); 21″ UXGA Flat Screen Monitor (1600×1200); Built-in Compact Flash with 1 GB for Image Capture; Image Processing Software with Measurement. Zoom Lenses and Accessories: MX-10C 10× Zoom High Magnification Lens; OL-700 700×-7000× Objective Lens for MX-10C ST-HU32 Motorized Z-axis focus block (0.05 micron steps) with XY table and Motor Control Unit; AS-B Back Light Kit with an External Light Source, Mirror, Condenser and Fiber Optic Light Guide.

FIGS. 1-4 show micrographs of emulsions A, B, C and D. The oil droplet size significantly decreases as the concentration of terpolymer of the invention increases.

FIG. 1 containing no terpolymer shows an oil droplet size varying from about 27 to 49 microns.

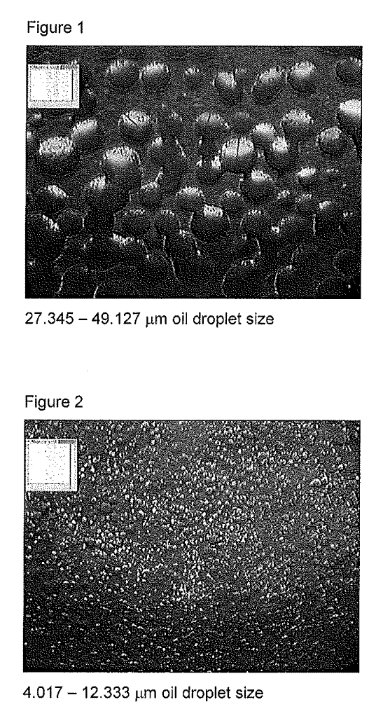

FIG. 2 containing 0.5 wt. % terpolymer as supplied (0.25% active) shows an oil droplet size varying from about 4 to about 12 microns.

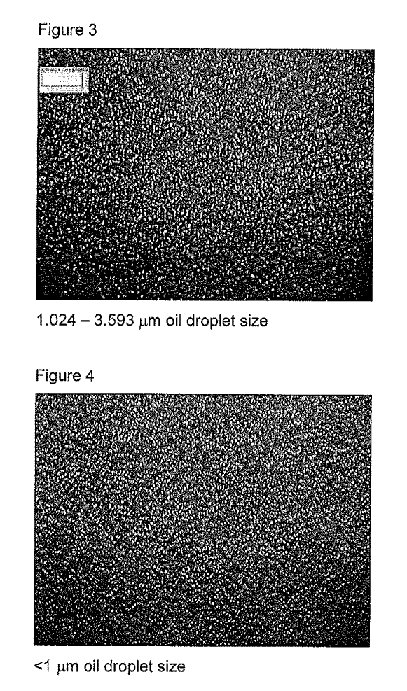

FIG. 3 containing 1.0 wt. % terpolymer as supplied (0.50% active) shows an oil droplet size varying from about 1 to about 3.5 microns.

FIG. 4 containing 2.0 wt. % terpolymer as supplied (1.0% active) shows an oil droplet size less than 1 micron.

Smaller oil droplet size contributes to the water-in-oil emulsion stability and non agglomeration of particulates. Smaller oil droplet size in oil-in-water emulsions also improves skin feel, important to any cosmetic formulation.

In addition, random terpolymer has improved the water resistant properties of oil-in-water emulsions.

### Example 26

### Transfer-Resistance Properties of the Lipstick Containing Random Terpolymer

### Materials & Equipment:

### Substrate—Vitro-Skin N-19, Lot 6169 (IMS, Inc.);

Commercial Lipstick—Milani Color Perfect Lipstick (Bing cherry Shade) is used as lipstick base
Stainless steel BYK Gardner 10 mil wet film Bird Applicator

### Scott C-Fold Towels (Kimberly-Clark)

### Random Terpolymer

Random Terpolymer is incorporated to the lipstick by thoroughly mixing it with the lipstick base (at ˜55° C.) and cooling it to the RT.

### Procedure:

This procedure simulates the transfer of a lipstick from the lips to another surface (for example, paper towel) that can occur during lipstick usage.

Vitro-Skin is pre-cut into 4.1 cm×4.1 cm pieces (a minimum of 3 pieces/sample is used) and placed in a closed, controlled-humidity chamber for 19-20 hours. The humidity in the chamber is regulated by 14.7% aqueous solution of glycerin. A piece of hydrated Vitro-Skin is placed on a parafilm-covered foam block. 0.033 g of the test article (lipstick) is applied evenly to the “skin topography” surface of the Vitro-Skin (this resulted in a product application dose of 2 mg/sq. cm). After application the substrate is allowed to air-dry for 5 minutes. Paper towel is pre-cut into 6 cm×6 cm pieces. Then a piece of paper towel is tared and positioned (rough side up) on a flat surface. Vitro Skin is placed on the paper towel, with side with applied lipstick facing down. An 8 cm×8 cm piece of parafilm is placed atop of the Vitro Skin, Scotch tape is used to secure the position of the parafilm. Bird Applicator is dragged over the parafilm in order to apply a standardized pressure on the substrate with applied lipstick. Afterwards the paper towel is weighed and the weight of the transferred lipstick is recorded. Larger weight corresponded to the increased transfer and the transfer resistance of a formulation is indicated by smaller weight values.

Results/Discussion: The results are presented in Table XIII below.

TABLE XIII

| | Weight of the Transferred Lipstick, g | | | | |
|---|---|---|---|---|---|
| Test Article | Trial 1 | Trial 2 | Trial 3 | Average | StDev |
| Commercial Lipstick | 0.0021 | 0.0027 | 0.0030 | 0.00260 | 0.00045 |
| (Base) | | | | | |
| Base with 5% Random | 0.0019 | 0.0018 | 0.0014 | 0.0017 | 0.00026 |
| Terpolymer of example | | | | | |
| 13 | | | | | |

The addition of Random Terpolymer significantly improved the transfer resistance of the lipstick base by reducing this parameter by 35%.

### Example 27

TABLE XIV
Water-In-Oil Emulsions

| | | A | B |
|---|---|---|---|
| Phase | Ingredients | % wt. | % wt. |
| Oil Phase | Glyceryl Dilaurate | 1.2 | 1.2 |
| | Isostearyl Neopentanoate | 15 | 15 |
| | PEG-30 Dipolyhydroxystearate | 2.9 | 2.9 |
| | C12-15 Alkyl Benzoate (And) Zinc | 28 | 28 |
| | Oxide (And) Polyhydroxystearic Acid | | |
| | (And) Triethoxycaprylylsilane | | |
| Water Phase | Di Water | 47.55 | 47.05 |
| | Example 13 terpolymer (as supplied) | 0 | 0.5 |
| | Propylene Glycol | 3 | 3 |
| | Magnesium Sulphate | 2.0 | 2.0 |
| Preservative | Diazolidinyl urea (and) Iodopropynyl | 0.35 | 0.35 |
| Phase | Carbamate (and) Propylene Glycol | | |
| Total | | 100.00 | 100.00 |

### Manufacturing Procedure

1. Mix Water Phase ingredients in the order listed and heat to 80° C.
2. Combine, heat and mix Oil Phase components to 80° C.
3. Add Water Phase to Oil Phase slowly with homogenization.
4. Cool to 40° C. with mixing, and add Preservative Phase with continuing mixing until system temperature is 30-35° C.

Water droplet size is reduced in Sample B.

1. A color cosmetic composition comprising
(a) at least one coloring agent, preferably a pigment,
(b) at least one random terpolymer of formula (I) 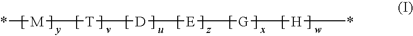
wherein
u, v, w, x, y, and z represent the percentage by weight that each repeating unit or derived monomer is contained within the terpolymer;
u, v, w, x, y, and z add up to total 100 weight percent relative to the total weight of the terpolymer;
y is from about 0 to about 40% by weight of the terpolymer;
v is from about 5% to about 75% by weight of the terpolymer;
u is from about 5% to about 80% by weight of the terpolymer;
z is from about 0% to about 60% by weight of the terpolymer;
X is from about 1% to about 50% by weight of the terpolymer;
w is from about 0% to about 50% by weight of the terpolymer;
* is a terminal group, for example, a catalyst residue;
M, T, D, E, G, and H are covalently bonded to each other;
M is derived from at least one monomer of formula (II) 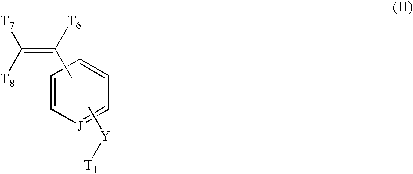
wherein T6, T7, and T8 are C1-C4 alkyl or hydrogen; Y is a direct bond, —O—, —S—, —N(H)— or —N(T1)-; T1 is hydrogen or C1-C4 alkyl; and J is a nitrogen or carbon atom;
T, D, and E are independently derived from at least one monomer of formula (III) 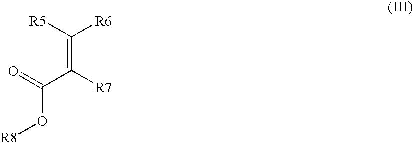
wherein R5, R6 and R7 may be the same or different and represent hydrogen or C1-C22 alkyl;
R8 is C1-C30 alkyl, C6-C15 cycloalkyl, or C6-C15 aryl; said substituted alkyl, said cycloalkyl or said aryl may also be substituted by one or more —OH and/or —NH2 groups; or said alkyl or said cycloalkyl may be interrupted by one or more —O— groups and/or N(H)— groups;
G is derived from at least one monomer comprising a heterocyclic group having at least one basic ring nitrogen atom or to which such a heterocyclic group is attached following polymerization;
H is derived from at least one monomer selected from the group consisting of toluene diisocyanate (all isomers), 4,4′-diphenylmethane diisocyanate, tolidine diisocyanate, dianisidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bis(2-methylisocyanatophenyl)methane, 4,4′-bisphenylene diisocyanate, 4,4′-bis(2-methoxyisocyanatophenyl)methane, 1-nitrophenyl-3,5-diisocyanate, 4,4′-diisocyanatodiphenyl ether, 3,3′-dichloro-4,4′-diisocyanatodiphenyl ether, 3,3′-dichloro-4,4′-diisocyanatodiphenyl methane, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,2-naphthalene diisocyanate, 4-chloro-1,2-naphthalene diisocyanate, 4-methyl-1,2-naphthalene diisocyanate, 1,5-naphthalene diisocyanate, 1,6-naphthalene diisocyanate, 1,7-naphthalene diisocyanate, 1,8-naphthalene diisocyanate, 4-chloro-1,8-naphthalene diisocyanate, 2,3-naphthalene diisocyanate, 2,7-naphthalene diisocyanate, 1,8-dinitro-2,7-naphthalene diisocyanate, 1-methyl-2,4-naphthalene diisocyanate, 1-methyl-5,7-naphthalene diisocyanate, 6-methyl-1,3-naphthalene diisocyanate, 7-methyl-1,3-naphthalene diisocyanate, 1,2-ethane diisocyanate, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, diisocyanates or a mixture thereof dimer acid derived diisocyanate obtained from dimerized linoleic acid, 4,4′-dicyclohexylmethane diisocyanate, isophorone diisocyanate, 3-isocyanatomethyl-3,5,5-trimethylcyclohexyl diisocyanate, lysine methyl ester diisocyanate, bis(2-isocyanatoethyl) fumarate bis(2-isocyanatoethyl) carbonate, m-tetramethylxylylene diisocyanate, acrylonitrile and mixtures thereof;
and
(c) optionally, other cosmetically acceptable ingredients,
with the proviso that T, D, and E are different from each other.

2. A composition according to claim 1 wherein for component b) formula (I) M is derived from at least one monomer selected from the group consisting of styrene, alpha-methylstyrene, 2-vinyltoluene, 3-vinyltoluene, 4-vinyltoluene, ethylvinylbenzene and mixtures thereof.

3. A composition according to claim 1 wherein for component b) formula (I) T, D, and E are independently derived from at least one monomer selected from the group consisting of methyl(meth)acrylate, ethyl(meth)acrylate, propyl(meth)acrylate, butyl(meth)acrylate, isobutyl(meth)acrylate, hexyl(meth)acrylate, cyclohexyl(meth)acrylate, 2-ethylhexyl(meth)acrylate, octyl(meth)acrylate, decyl(meth)acrylate, dodecyl (meth)acrylate, 2-ethyl hexyl(meth)acrylate, dimethyl aminoethyl(meth)acrylate, isobornyl(meth)acrylate, stearyl(meth)acrylate, behenyl(meth)acrylate, polypropylene glycol mono(meth)acrylate, glycidyl(meth)acrylate, polyethylene glycol mono(meth)acrylate, EO-PO-mono(meth)acrylate and mixtures thereof.

4. A composition according to claim 1 wherein for component b formula (I) G is selected from the group consisting of vinylimidazole, 2-vinylpyridine, 4-vinylpyridine, 2-methyl-N-vinylimidazole, vinylpyrrolidone, vinylcarbazole and mixtures thereof.

5. A composition according to claim 1 wherein for component b formula (I) G is selected from the group consisting of 1-(2-hydroxyethyl)-pyrrolidine, 2-(1-pyrrolidyl)-ethylamine, 2-(1-piperidyl)-ethylamine, 1-(2-hydroxyethyl)-piperidine, 1-(2-aminopropyl)-piperidine, N-(2-hydroxyethyl)-hexamethylenimine, 4-(2-hydroxyethyl)-morpholine, 2-(4-morpholinyl)-ethylamine, 4-(3-aminopropyl)-morpholine, 1-(2-hydroxyethyl)-piperazine, 1-(2-aminoethyl)-piperazine, 1-(2-hydroxyethyl)-2-alkylimidazoline, 1-(3-aminopropyl)-imidazole, (2-aminoethyl)-pyridine, (2-hydroxyethyl)-pyridine, (3-hydroxypropyl)-pyridine, (hydroxymethyl)-pyridine, N-methyl-2-hydroxy-methyl-piperidine, 1-(2-hydroxyethyl)-imidazole, 2-amino-6-methoxybenzothiazole, 4-aminomethyl-pyridine, 4-amino-2-methoxypyrimidine, 2-mercaptopyrimidine, 2-mercapto-benzimidazole, 3-mercapto-1,2,4-triazole, 3-amino-1,2,4-triazole, 2-isopropyl-imidazole, 2-ethyl-imidazole, 4-methyl-imidazole, 2-methyl-imidazole, 2-ethyl-4-methyl-imidazole, 2-phenyl-imidazole, 4-nitro-imidazole and mixtures thereof.

6. A composition according to claim 1 wherein for component b) formula (I) H is derived from at least one monomer selected from the group consisting of toluene diisocyanate, 4,4′-diphenylmethane diisocyanate, tolidine diisocyanate, m-xylylene diisocyanate, p-phenylene diisocyanate, m-phenylene diisocyanate, 1-chloro-2,4-phenylene diisocyanate, 3,3′-dimethyl-4,4′-bisphenylene diisocyanate, 4,4′-bisphenylene diisocyanate, 4,4′-bis(2-methoxyisocyanatophenyl)methane, 4,4′-diisocyanatodiphenyl ether, 4,4′-diisocyanatodibenzyl, 3,3′-dimethoxy-4,4′-diisocyanatodiphenyl, 2,2′-dimethyl-4,4′-diisocyanatodiphenyl, 2,2′-dichloro-5,5′-dimethoxy-4,4′-diisocyanatodiphenyl, 3,3′-dichloro-4,4′-diisocyanatodiphenyl, 1,3-propane diisocyanate, 1,4-butane diisocyanate, 2-chloropropane-1,3-diisocyanate, pentamethylene diisocyanate, propylene-1,2-diisocyanate, 1,8-octane diisocyanate, 1,10-decane diisocyanate, 1,12-dodecane diisocyanate, 1,16-hexadecane diisocyanate 1,3- and 1,4-cyclohexane diisocyanate, 1,6-hexamethylene diisocyanate, 2,2,4- and 2,4,4-trimethylhexamethylene diisocyanate, 4,4′-dicyclohexylmethane diisocyanate, isophorone diisocyanate, 3-isocyanatomethyl-3,5,5-trimethylcyclohexyl diisocyanate, lysine methyl ester diisocyanate, m-tetramethylxylylene diisocyanate and mixtures thereof.

7. A composition according to claim 1 wherein
y is from about 0.1 to about 35% by weight of the terpolymer of formula (I);
v is from about 5% to about 70% by weight of the terpolymer of formula (I);
u is from about 5% to about 75% by weight of the terpolymer of formula (I);
z is from about 0.1% to about 50% by weight of the terpolymer of formula (I);
x is from about 1% to about 40% by weight of the terpolymer of formula (I); and
w is from about 01% to about 45% by weight of the terpolymer of formula (I).

8. A composition according to claim 7 wherein
y is from about 1 to about 30% by weight of the terpolymer of formula (I);
v is from about 5% to about 60% by weight of the terpolymer of formula (I);
u is from about 5% to about 65% by weight of the terpolymer of formula (I);
z is from about 1% to about 50% by weight of the terpolymer of formula (I);
x is from about 1% to about 30% by weight of the terpolymer of formula (I); and
w is from about 1% to about 40% by weight of the terpolymer of formula (I).

9. A composition according to claim 8 wherein
y is from about 5 to about 20% by weight of the terpolymer of formula (I);
v is from about 10% to about 60% by weight of the terpolymer of formula (I);
u is from about 5% to about 60% by weight of the terpolymer of formula (I);
z is from about 1% to about 40% by weight of the terpolymer of formula (I);
x is from about 5% to about 25% by weight of the terpolymer of formula (I); and
w is from about 5% to about 30% by weight of the terpolymer of formula (I).

10. A composition according to claim 1 wherein component b) formula (I) has a weight average molecular weight of from about 500 to about 1 million Daltons.

11. A composition according to claim 10 wherein component b) formula (I) has a weight average molecular weight of from about 500 to about 500,000 Daltons.

12. A composition according to claim 11 wherein component b) formula (I) has a weight average molecular weight of from about 500 to about 100,000 Daltons.

13. A composition according to claim 12 wherein component b) formula (I) has a weight average molecular weight of from about 1000 to about 75,000 Daltons.

14. A composition according to claim 1 wherein component b) formula (I) is present in an amount from about 0.01 weight percent to about 50 weight percent based on the weight of the total composition.

15. A composition according to claim 14 wherein component b) formula (I) is present in an amount from about 0.1 weight percent to about 25 weight percent based on the weight of the total composition.

16. A composition according to claim 15 wherein component b) formula (I) is present in an amount from about 0.1 weight percent to about 10 weight percent based on the weight of the total composition.

17. A composition according to claim 1 wherein component a) is an organic or inorganic pigment.

18. A composition according to claim 17 wherein said inorganic pigment is selected from the group consisting of alumina, chromium-cobalt-aluminum oxide, ferric ammonium citrate, calcium carbonate, Iron oxides (synthetic and natural), ferric ammonium ferrocyanide, chromium hydroxide green, bismuth oxychloride, chromium oxide green, mica, titanium dioxide, aluminum powder, bronze powder, copper powder, ultramarines, manganese violet, zinc oxide and mixtures thereof.

19. A composition according to claim 17 wherein said organic pigment is selected from the group consisting of FD&C Blue No. 1 (CI No. 42090), FD&C Green No. 3 (CI No. 42053), FD&C Red No. 3 (CI No. 45430), FD&C Red No. 40 (CI No. 16035), FD&C Yellow No. 5 (CI No. 19140), FD&C Yellow No. 6 (CI No. 15985), D&C Blue No. 4 (CI No. 42090), D&C Green No. 5 (CI No. 61570), D&C Green No. 6 (CI No. 61565), D&C Yellow No. 10 (CI No. 47005), Ext D&C Yellow No. 7 (CI No. 10315), D&C Brown No. 1 (CI No. 20170), D&C Orange No. 4 (CI No. 15510), D&C Orange No. 5 (CI No. 45425), D&C Orange 17 (CI No. 12075), D&C Red No. 6 (CI No. 15850), D&C Red No. 7 (CI No. 15850), D&C Red No. 8 (CI No. 15585), D&C Red No. 9 (CI No. 15585), D&C Red No. 17 (CI No. 26100), D&C Red No. 19 (CI No. 45170), D&C Red No. 21 (CI No. 45380A), D&C Red No. 27 (CI No. 45410A), D&C Red No. 28 (CI No. 45410), D&C Red No. 30 (CI No. 73360), D&C Red No. 31 (CI No, 15800), D&C red No, 33 (CI No. 17200), D&C Red No. 34 (CI No. 15880), D&C Red No. 36 (CI No. 12085), D&C red No. 37 (CI No. 45170B), D&C Violet No. 2 (CI No. 60725), Ext D&C Violet No. 2 (CI No. 60730), D&C Lakes-stratight colorant extended on substratum of alumina, blanc fixe, gloss, white, clay, titanium dioxide, zinc oxide, talc, rosin, aluminum benzoate, calcium carbonate, or any combination of two or more of these, Ext. D&C Lakes-stratight colorant extended on substratum of alumina, blanc fixe, gloss, white, clay, titanium dioxide, zinc oxide, talc, rosin, aluminum benzoate, calcium carbonate, or any combination of two or more of these and mixtures thereof.
and mixtures thereof.

20. A composition according to claim 17 wherein the component a) is a particle ranging in size from about 0.0001 to about 200 microns.

21. A composition according to claim 1 wherein the color cosmetic composition further contains water.

22. A composition according to claim 1 wherein the color cosmetic composition is an oil-in-water or water-in-oil emulsion.

23. A composition according to claim 21 wherein the water present in the color composition ranges from about 1 to about 80 weight % of the composition.

24. A day cream, powder cream, face powder (loose or pressed), rouge, cream make-up, eyeshadow, mascara, eyeliner, eyebrow pencil, lipstick, lip gloss, lip contour pencils and nail varnish comprising the compositions according to claim 1.

25. A method of increasing the smudge resistance of a cosmetic color composition on skin or hair wherein said method comprises incorporating into said compositions a composition according to claim 1.

26. A method of improving the water resistance of a color cosmetic on skin or hair which method comprises applying to said skin or said hair an effective amount of the color cosmetic composition according to claim 1.

27. A method of reducing the oil droplet size of a cosmetic oil-in-water cosmetic emulsion which method comprises the steps of adding a random terpolymer of formula (I) to an oil, a water phase or to a formed oil-in-water cosmetic emulsion.
